(19) United States
(12) Patent Application Publication
(10) Pub. No.: US 2008/0015222 A1
(43) Pub. Date: Jan. 17, 2008

(54) New Heterocyclic Amides

(75) Inventors: Yevgeni Besidski, Sodertalje (SE); Inger Kers, Sodertalje (SE); Martin Nylof, Sodertalje (SE); Andis Slaitas, Riga (LV)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/575,635

(22) PCT Filed: Sep. 19, 2005
(86) PCT No.: PCT/SE05/01364

§ 371 Date: Mar. 20, 2007

(30) Foreign Application Priority Data
Sep. 21, 2004 (SE) 0402284-4

(51) Int. Cl. A61K 31/4184; A61K 31/4174; A61P 1/00; A61P 11/00; A61P 29/00; C07D 235/06; C07D 401/04

(52) U.S. Cl. 514/314; 514/394; 546/273.4; 548/343.5

(57) ABSTRACT

The present invention relates to new compounds 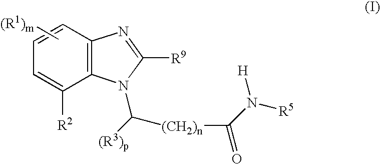 or salts, solvates or solvated salts thereof, processes for their preparation and to new intermediates used in the preparation thereof, pharmaceutical compositions containing said compounds and to the use of said compounds in therapy.

### FIELD OF THE INVENTION

The present invention relates to new compounds, to pharmaceutical compositions containing said compounds and to the use of said compounds in therapy. The present invention further relates to processes for the preparation of said compounds and to new intermediates used in the preparation thereof.

### BACKGROUND OF THE INVENTION

Pain sensation in mammals is due to the activation of the peripheral terminals of a specialized population of sensory neurons known as nociceptors. Capsaicin, the active ingredient in hot peppers, produces sustained activation of nociceptors and also produces a dose-dependent pain sensation in humans. Cloning of the vanilloid receptor 1 (VR1 or TRPV1) demonstrated that VR1 is the molecular target for capsaicin and its analogues. (Caterina, M. J., Schumacher, M. A., et. al. Nature (1997) v. 389 p 816-824). Functional studies using VR1 indicate that it is also activated by noxious heat, tissue acidification) and other inflammatory mediators (Tominaga, M., Caterina, M. J. et. al. Neuron (1998) v. 21, p. 531-543). Expression of VR1 is also regulated after peripheral nerve damage of the type that leads to neuropathic pain. These properties of VR1 make it a highly relevant target for pain and for diseases involving inflammation. While agonists of the VR1 receptor can act as analgesics through nociceptor destruction, the use of agonists, such as capsaicin and its analogues, is limited due to their pungency, neurotoxicity and induction of hypothermia. Instead, agents that block the activity of VR1 should prove more useful. Antagonists would maintain the analgesic properties, but avoid pungency and neurotoxicity side effects. Compounds with VR1 inhibitor activity are believed to be of potential use for the treatment and/or prophylaxis of disorders such as pain, especially that of inflammatory or traumatic origin such as arthritis, ischaemia, cancer, fibromyalgia, low back pain and post-operative pain (Walker et al J Pharmacol Exp Ther. (2003) Jan; 304(1):56-62). In addition to this visceral pains such as chronic pelvic pain, cystitis, irritable bowel syndrome (IBS), pancreatitis and the like, as well as neuropathic pain such as sciatia, diabetic neuropathy, HIV neuropathy, multiple sclerosis, and the like (Walker et al ibid, Rashid et al J Pharmacol Exp Ther. (2003) Mar; 304(3):940-8), are potential pain states that could be treated with VR1 inhibition These compounds are also believed to be potentially useful for inflammatory disorders like asthma, cough, inflammatory bowel disease (IBD) (Hwang and Oh Curr Opin Pharmacol (2002) Jun.; 2(3):235-42). Compounds with VR1 blocker activity are also useful for itch and skin diseases like psoriasis and for gastro-esophageal reflux disease (GERD), emesis, cancer, urinary incontinence and hyperactive bladder (Yiangou et al BJU Int (2001) Jun.; 87(9):774-9, Szallasi Am J Clin Pathol (2002) 118: 110-21). VR1 inhibitors are also of potential use for the treatment and/or prophylaxis of the effects of exposure to VR1 activators like capsaicin or tear gas, acids or heat (Szallasi ibid.

A further potential use relates to the treatment of tolerance to VR1 activators. VR1 inhibitors may also be useful in the treatment of interstitial cystitis and pain related to interstitial cystitis.

### DETAILED DESCRIPTION OF THE INVENTION

The object of the present invention is to provide compounds exhibiting an inhibitory activity at the vanilloid receptor 1 (VR1).

The present invention provides compounds of formula I 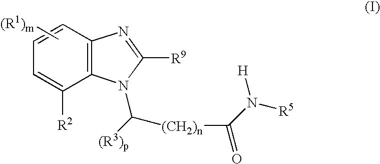
wherein:
R¹ is H, NO₂, halo, NR⁶R⁷, C₁₋₆alkyl, C₂₋₆alkenyl, C₂₋₆alkynyl, C₁₋₆haloalkyl, C₁₋₆haloalkylO, R⁶OC₀₋₆alkyl, R⁶CO, R⁶OCO or CONR⁶R⁷;
m is 0, 1, 2 or 3;
R² is H, NO₂, halo, NR⁶R⁷, C₁₋₆alkyl, C₂₋₆alkenyl, C₂₋₆alkynyl, C₁₋₆haloalkyl, C₁₋₆haloalkylO, cyano, R⁶OC₀₋₆alkyl, R⁶CO, R⁶OCO, R⁶CONR⁷, R⁶R⁷NCO, R⁵SO₂, R⁸SO₂HN, arylC₀₋₆alkyl or heteroarylC₀₋₆alkyl;
R³ and R⁹ are each independently H or C₁₋₄alkyl;
R² and R³ optionally form a ring;
p is 0, 1 or 2;
n is 0, 2, 3 or 4;
R⁵ is C₁₋₁₀alkyl, C₆₋₁₀arylC₀₋₆alkyl, C₃₋₇cycloalkylC₀₋₆alkyl or C₅₋₆heteroarylC₀₋₆alkyl, whereby any aryl, heteroaryl or cycloalkyl may be fused with aryl, heteroaryl, C₃₋₇cycloalkyl or C₃₋₇heterocycloalkyl, and which R⁵ may be substituted with one or more A;
A is H, OH, NO₂, cyano, R⁶CO, R⁶O(CO), halo, C₁₋₆alkyl, NR⁶R⁷, C₁₋₆haloalkyl, C₁₋₆haloalkylO, R⁶OC₀₋₆alkyl, hydroxyC₁₋₆alkyl, R⁸SO₂, R⁸SO₂HN, C₅₋₆arylO or CONR⁶R⁷;
R⁶ and R⁷ are each independently H or C₁₋₆alkyl; and
R⁸ is NR⁶R⁷ or C₁₋₄alkyl
or salts, solvates or solvated salts thereof.

Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, t-pentyl, neo-pentyl, n-hexyl or i-hexyl, t-hexyl. The term C₁₋₃ alkyl having 1 to 3 carbon atoms and may be methyl, ethyl, n-propyl, i-propyl or tert-butyl.

The term ‘C₀’ means a bond or does not exist. For example when R³ is C₀alkyl, R³ is a bond and “arylC₀alkyl” is equivalent with “aryl”, “C₂alkylOC₀alkyl” is equivalent with “C₂alkylO”.

In this specification, unless stated otherwise, the term “alkenyl” includes both straight and branched chain alkenyl groups. The term “C₂₋₆alkenyl” having 2 to 6 carbon atoms and one or two double bonds, may be, but is not limited to vinyl, allyl, propenyl, butenyl, crotyl, pentenyl, or hexenyl, and a butenyl group may for example be buten-2-yl, buten-3-yl or buten-4-yl.

In this specification, unless stated otherwise, the term “alkynyl” includes both straight and branched chain alkynyl groups. The term “C₂₋₆alkynyl” having 2 to 6 carbon atoms and one or two trippel bonds, may be, but is not limited to etynyl, propargyl, pentynyl or hexynyl and a butynyl group may for example be butyn-3-yl or butyn-4-yl.

In this specification, unless stated otherwise, the term “cycloalkyl” refers to an optionally substituted, saturated cyclic hydrocarbon ring system. The term “C₃₋₇cycloalkyl” may be cyclopropyl, cyclobutyl, cyclopentyl, cyclohexyl or cycloheptyl.

The term “heterocycloalkyl” denotes a 3- to 7-membered, non-aromatic, partially or completely saturated hydrocarbon group, which contains one ring and at least one heteroatom. Examples of said heterocycle include, but are not limited to pyrrolidinyl, pyrrolidonyl, piperidinyl, piperazinyl, morpholinyl, oxazolyl, 2-oxazolidonyl or tetrahydrofuranyl.

In this specification, unless stated otherwise, the term “aryl” refers to an optionally substituted monocyclic or bicyclic hydrocarbon unsaturated aromatic ring system. Examples of “aryl” may be, but are not limited to phenyl and naphthyl.

In this specification, unless stated otherwise, the term “heteroaryl” refers to an optionally substituted monocyclic or bicyclic unsaturated aromatic ring system containing at least one heteroatom selected independently form N, O or S. Examples of “heteroaryl” may be, but are not limited to pyridyl, pyrrolyl, furyl, thienyl, imidazolyl, oxazolyl, isoxazolyl, thiazolyl, pyrazolyl, benzofuryl, indolyl, isoindolyl, benzimidazolyl, pyridazinyl, pyrimidinyl, pyrazinyl, tetrazolyl, triazolyl or oxazolyl.

In this specification, unless stated otherwise, the terms “arylalkyl” and “heteroarylalkyl” refer to a substituent that is attached via the alkyl group to an aryl or heteroaryl group. In this specification, unless stated otherwise, the terms “halo” and “halogen” may be fluoro, iodo, chloro or bromo.

In this specification, unless stated otherwise, the term “haloalkyl” means an alkyl group as defined above, which is substituted with halo as defined above. The term “C₁₋₆haloalkyl” may include, but is not limited to fluoromethyl, difluoromethyl, trifluoromethyl, fluoroethyl, difluoroethyl or bromopropyl. The term “C₁₋₆haloalkylO” may include, but is not limited to fluoromethoxy, difluoromethoxy, trifluoromethoxy, fluoroethoxy or difluoroethoxy.

The present invention provides compounds selected from the group consisting of
N-{3-[2-(dimethylamino)ethoxy]phenyl}-2-(7-nitro-1H-benzimidazol-1-yl)acetamide,
N-[3-(methoxymethyl)phenyl]-2-(7-nitro-1H-benzimidazol-1-yl)acetamide,
N-(1,3-dihydro-2-benzofuran-5-yl)-2-(7-nitro-1H-benzimidazol-1-yl)acetamide,
N-[3-methoxy-5-(methoxymethyl)phenyl]-2-(7-nitro-1H-benzimidazol-1-yl)acetamide,
N-[3-(methoxymethyl)-5-(trifluoromethyl)phenyl]-2-(7-nitro-1H-benzimidazol-1-yl)acetamide,
2-(7-acetyl-1H-benzimidazol-1-yl)-N-[3-(methoxymethyl)-5-(trifluoromethyl)phenyl]acetamide,
N-[3-cyano-5-(trifluoromethyl)phenyl]-2-(7-nitro-1H-benzimidazol-1-yl)acetamide,
N-[3-acetyl-5-(trifluoromethyl)phenyl]-2-(7-nitro-1H-benzimidazol-1-yl)acetamide,
2-(7-acetyl-1H-benzimidazol-1-yl)-N-[3-acetyl-5-(trifluoromethyl)phenyl]acetamide,
2-(7-acetyl-1H-benzimidazol-1-yl)-N-[3-cyano-5-(trifluoromethyl)phenyl]acetamide,
N-[3-(1-methoxyethyl)phenyl]-2-(7-nitro-1H-benzimidazol-1-yl)acetamide,
2-(7-chloro-6-methoxy-1H-benzimidazol-1-yl)-N-(2,3-dihydro-1H-inden-5-yl)acetamide,
N-[3-(2-methoxyethoxy)phenyl]-2-(7-nitro-1H-benzimidazol-1-yl)acetamide,
N-[3-methoxy-5-(2-methoxyethoxy)phenyl]-2-(7-nitro-1H-benzimidazol-1-yl)acetamide,
N-[3-(2-methoxyethoxy)-5-(trifluoromethyl)phenyl]-2-(7-nitro-1H-benzimidazol-1-yl)acetamide,
N-[3-methoxy-5-(tetrahydrofuran-2-ylmethoxy)phenyl]-2-(7-nitro-1H-benzimidazol-1-yl)acetamide,
N-[3-methoxy-5-(tetrahydrofuran-3-yloxy)phenyl]-2-(7-nitro-1H-benzimidazol-1-yl)acetamide,
N-[3-methoxy-5-(trifluoromethyl)phenyl]-5,6-dihydro-4H-imidazo[4,5,1-ij]quinoline-4-carboxamide,
2-(7-amino-1H-benzimidazol-1-yl)-N-(4-tert-butylbenzyl)acetamide,
N-(4-tert-butylbenzyl)-2-(7-iodo-1H-benzimidazol-1-yl)acetamide,
2-[7-(1-hydroxy-1-methylethyl)-1H-benzimidazol-1-yl]-N-[3-methoxy-5-(trifluoromethyl)phenyl]acetamide,
N-(4-tert-butylbenzyl)-2-[7-(1-hydroxyethyl)-1H-benzimidazol-1-yl]acetamide,
N-(2,3-dihydro-1H-inden-5-yl)-2-(7-pyridin-2-yl-1H-benzimidazol-1-yl)acetamide,
N-(4-tert-butylbenzyl)-2-(7-pyridin-2-yl-1H-benzimidazol-1-yl)acetamide,
2-(7-acetyl-1H-benzimidazol-1-yl)-N-(4-tert-butylbenzyl)acetamide,
2-(7-acetyl-1H-benzimidazol-1-yl)-N-[3-methoxy-5-(trifluoromethyl)phenyl]acetamide,
2-(7-nitro-1H-benzimidazol-1-yl)-N-(3,4,5-trifluorophenyl)acetamide,
N-(4-tert-butylbenzyl)-2-(7-cyano-1H-benzimidazol-1-yl)acetamide,
2-(7-cyano-1H-benzimidazol-1-yl)-N-(3,4,5-trimethoxyphenyl)acetamide,
N-(4-bromo-2-fluorophenyl)-2-(7-cyano-1H-benzimidazol-1-yl)acetamide,
2-(7-cyano-1H-benzimidazol-1-yl)-N-(3,4-difluorophenyl)acetamide,
2-(7-cyano-1H-benzimidazol-1-yl)-N-(3-ethoxyphenyl)acetamide,
2-(7-nitro-1H-benzimidazol-1-yl)-N-(3,4,5-trimethoxybenzyl)acetamide,
N-(3,4-difluorobenzyl)-2-(7-nitro-1H-benzimidazol-1-yl)acetamide,
N-[2-(4-methoxyphenyl)ethyl]-2-(7-nitro-1H-benzimidazol-1-yl)acetamide,
N-[2-(3-fluorophenyl)ethyl]-2-(7-nitro-1H-benzimidazol-1-yl)acetamide,
N-[2-(3-methoxyphenyl)ethyl]-2-(7-nitro-1H-benzimidazol-1-yl)acetamide,
2-(7-nitro-1H-benzimidazol-1-yl)-N-{2-[3-(trifluoromethyl)phenyl]ethyl}acetamide,
N-[2-(3,4-dimethoxyphenyl)ethyl]-2-(7-nitro-1H-benzimidazol-1-yl)acetamide,
2-(7-acetyl-1H-benzimidazol-1-yl)-N-(3,4,5-trimethoxyphenyl)acetamide,
2-(7-acetyl-1H-benzimidazol-1-yl)-N-(3,4-difluorophenyl)acetamide,
2-(7-acetyl-1H-benzimidazol-1-yl)-N-(3,5-dimethoxyphenyl)acetamide,
N-[2-(3,5-dimethoxyphenyl)ethyl]-2-(7-nitro-1H-benzimidazol-1-yl)acetamide,
N-(2,3-dihydro-1H-inden-2-yl)-2-(7-nitro-1H-benzimidazol-1-yl)acetamide,
N-[2-(5-bromo-2-methoxyphenyl)ethyl]-2-(7-nitro-1H-benzimidazol-1-yl)acetamide,
N-[1-(4-chlorobenzyl)-2-hydroxyethyl]-2-(7-nitro-1H-benzimidazol-1-yl)acetamide,
N-(2-hydroxy-2-phenylethyl)-2-(7-nitro-1H-benzimidazol-1-yl)acetamide,
1-{2-[(3,5-dimethoxyphenyl)amino]-2-oxoethyl}-1H-benzimidazole-7-carboxylic acid,
1-[2-(2,3-dihydro-1H-inden-5-ylamino)-2-oxoethyl]-1H-benzimidazole-7-carboxylic acid,
N-(3,5-dimethoxyphenyl)-2-[7-(hydroxymethyl)-1H-benzimidazol-1-yl]acetamide,
1-{2-[(3,5-dimethoxyphenyl)amino]-2-oxoethyl}-N-ethyl-1H-benzimidazole-7-carboxamide,
1-{2-[(3,5-dimethoxyphenyl)amino]-2-oxoethyl}-N-methyl-1H-benzimidazole-7-carboxamide,
1-{2-[(3,5-dimethoxyphenyl)amino]-2-oxoethyl}-N,N-dimethyl-1H-benzimidazole-7-carboxamide,
1-{2-[(3,5-dimethoxyphenyl)amino]-2-oxoethyl}-N-methoxy-1H-benzimidazole-7-carboxamide,
ethyl 1-{2-[(3,5-dimethoxyphenyl)amino]-2-oxoethyl}-1H-benzimidazole-7-carboxylate, ethyl 1-{2-[(4-tert-butylbenzyl)amino]-2-oxoethyl}-1H-benzimidazole-7-carboxylate, ethyl 1-[2-(2,3-dihydro-1H-inden-5-ylamino)-2-oxoethyl]-11H-benzimidazole-7-carboxylate,
N-(4-tert-butylbenzyl)-2-[7-(dimethylamino)-1H-benzimidazol-1-yl]acetamide,
N-(4-methoxy-2-naphthyl)-2-(7-nitro-1H-benzimidazol-1-yl)acetamide,
2-(1H-benzimidazol-1-yl)-N-(4-tert-butylbenzyl)acetamide, and
2-(1H-benzimidazol-1-yl)-N-(2,3-dihydro-1H-inden-5-yl)acetamide, or salts, solvates or solvated salts thereof.

The present invention further provides compounds selected from the group consisting of
N-(3,5-dimethoxyphenyl)-2-(7-ethynyl-1H-benzimidazol-1-yl)acetamide,
N-(3,5-dimethoxyphenyl)-2-(7-prop-1-yn-1-yl-1H-benzimidazol-1-yl)acetamide,
N-(3,5-dimethoxyphenyl)-2-[7-(1H-1,2,3-triazol-4-yl)-1H-benzimidazol-1-yl]acetamide,
N-(3,5-dimethoxyphenyl)-2-[7-(1-methyl-11H-1,2,3-triazol-4-yl)-1H-benzimidazol-1-yl]acetamide,
N-(3,5-dimethoxyphenyl)-2-[7-(1-methyl-1H-tetrazol-5-yl)-11H-benzimidazol-1-yl]acetamide,
2-(7-ethyl-1H-benzimidazol-1-yl)-N-[3-methoxy-5-(trifluoromethyl)phenyl]acetamide,
2-[7-(2-hydroxyethyl)-1H-benzimidazol-1-yl]-N-[3-methoxy-5-(trifluoromethyl)phenyl]acetamide,
2-[7-(2-hydroxy-1-methylethyl)-1H-benzimidazol-1-yl]-N-[3-methoxy-5-(trifluoromethyl)phenyl]acetamide,
N-[3-methoxy-5-(trifluoromethyl)phenyl]-2-(7-vinyl-1H-benzimidazol-1-yl)acetamide,
2-(7-isopropenyl-1H-benzimidazol-1-yl)-N-[3-methoxy-5-(trifluoromethyl)phenyl]acetamide,
2-(7-isopropyl-1H-benzimidazol-1-yl)-N-[3-methoxy-5-(trifluoromethyl)phenyl]acetamide,
N-(3,5-dimethoxyphenyl)-2-(7-methoxy-1H-benzimidazol-1-yl)acetamide,
N-(3,5-dimethoxyphenyl)-2-(7-ethoxy-1H-benzimidazol-1-yl)acetamide,
N-(3,5-dimethoxyphenyl)-2-(7-isopropoxy-1H-benzimidazol-1-yl)acetamide,
2-(7-tert-butoxy-1H-benzimidazol-1-yl)-N-(3,5-dimethoxyphenyl)acetamide,
N-(3,5-dimethoxyphenyl)-2-[7-(trifluoromethoxy)-1H-benzimidazol-1-yl]acetamide
N-(3,5-dimethoxyphenyl)-2-[7-(methylsulfinyl)-1H-benzimidazol-1-yl]acetamide,
2-[7-(difluoromethyl)-1H-benzimidazol-1-yl]-N-(3,5-dimethoxyphenyl)acetamide,
2-[7-(cyanomethyl)-1H-benzimidazol-1-yl]-N-(3,5-dimethoxyphenyl)acetamide,
2-[7-(aminomethyl)-1H-benzimidazol-1-yl]-N-(3,5-dimethoxyphenyl)acetamide
N-(3,5-dimethoxyphenyl)-2-{7-[(dimethylamino)methyl]-1H-benzimidazol-1-yl}acetamide,
2-(7-cyclopropyl-1H-benzimidazol-1-yl)-N-(3,5-dimethoxyphenyl)acetamide,
2-(7-cyclobutyl-11H-benzimidazol-1-yl)-N-(3,5-dimethoxyphenyl)acetamide,
N-(3,5-dimethoxyphenyl)-2-[7-(methoxymethyl)-1H-benzimidazol-1-yl]acetamide,
N-(1-isopropyl-1H-benzimidazol-5-yl)-2-(7-nitro-1H-benzimidazol-1-yl)acetamide,
2-(7-fluoro-1H-benzimidazol-1-yl)-N-(1-isopropyl-1H-benzimidazol-5-yl)acetamide,
2-(7-cyano-1H-benzimidazol-1-yl)-N-(1-isopropyl-1H-benzimidazol-5-yl)acetamide,
N-(1-tert-butyl-1H-benzimidazol-5-yl)-2-(7-nitro-1H-benzimidazol-1-yl)acetamide,
N-(1-tert-butyl-2-methyl-1H-benzimidazol-5-yl)-2-(7-nitro-1H-benzimidazol-1-yl)acetamide,
N-(1-isopropyl-2-methyl-1H-benzimidazol-5-yl)-2-(7-nitro-1H-benzimidazol-1-yl)acetamide,
2-(7-cyano-1H-benzimidazol-1-yl)-N-(1-isopropyl-2-methyl-1H-benzimidazol-5-yl)acetamide,
N-(1-tert-butyl-2-methyl-1H-benzimidazol-5-yl)-2-(7-cyano-1H-benzimidazol-1-yl)acetamide,
N-(1-tert-butyl-1H-benzimidazol-5-yl)-2-(7-cyano-1H-benzimidazol-1-yl)acetamide,
N-(1-tert-butyl-1H-benzimidazol-5-yl)-2-(7-fluoro-1H-benzimidazol-1-yl)acetamide,
N-(1-tert-butyl-2-methyl-1H-benzimidazol-5-yl)-2-(7-fluoro-1H-benzimidazol-1-yl)acetamide,
2-(7-fluoro-1H-benzimidazol-1-yl)-N-(1-isopropyl-2-methyl-1H-benzimidazol-5-yl)acetamide,
N-[1-isopropyl-7-(trifluoromethyl)-1H-benzimidazol-5-yl]-2-(7-nitro-1H-benzimidazol-1-yl)acetamide,
2-(7-fluoro-1H-benzimidazol-1-yl)-N-[1-isopropyl-7-(trifluoromethyl)-1H-benzimidazol-5-yl]acetamide,
2-(7-cyano-1H-benzimidazol-1-yl)-N-[1-isopropyl-7-(trifluoromethyl)-1H-benzimidazol-5-yl]acetamide,
N-2-naphthyl-2-(7-nitro-1H-benzimidazol-1-yl)acetamide,
2-(7-cyano-1H-benzimidazol-1-yl)-N-2-naphthylacetamide,
2-(7-fluoro-1H-benzimidazol-1-yl)-N-2-naphthylacetamide,
2-(7-cyano-1H-benzimidazol-1-yl)-N-(4-methoxy-2-naphthyl)acetamide,
2-(7-fluoro-1H-benzimidazol-1-yl)-N-(4-methoxy-2-naphthyl)acetamide,
N-[3-methoxy-5-(tetrahydro-2H-pyran-2-ylmethoxy)phenyl]-2-(7-nitro-1H-benzimidazol-1-yl)acetamide,
N-[3-(2-isopropoxyethoxy)-5-methoxyphenyl]-2-(7-nitro-1H-benzimidazol-1-yl)acetamide,
N-[3,5-bis(2-ethoxyethoxy)phenyl]-2-(7-nitro-1H-benzimidazol-1-yl)acetamide,
N-{3-methoxy-5-[2-(2-oxopyrrolidin-1-yl)ethoxy]phenyl}-2-(7-nitro-1H-benzimidazol-1-yl)acetamide,
N-[3-methoxy-5-(3-morpholin-4-ylpropoxy)phenyl]-2-(7-nitro-1H-benzimidazol-1-yl)acetamide,
N,N-diethyl-2-(3-methoxy-5-{[(7-nitro-1H-benzimidazol-1-yl)acetyl]amino}phenoxy)acetamide,
N-{3-methoxy-5-[(1-methylpiperidin-2-yl)methoxy]phenyl}-2-(7-nitro-1H-benzimidazol-1-yl)acetamide,
N-{3-[2-(1H-imidazol-1-yl)ethoxy]-5-methoxyphenyl}-2-(7-nitro-1H-benzimidazol-1-yl)acetamide, and
N-{3-methoxy-5-[(1-methyl-1H-imidazol-5-yl)methoxy]phenyl}-2-(7-nitro-1H-benzimidazol-1-yl)acetamide,
   or salts, solvates or solvated salts thereof.

The present invention relates to the compounds of the invention as hereinbefore defined as well as to the salts, solvates or solvated salts thereof. Salts for use in pharmaceutical compositions will be pharmaceutically acceptable salts, but other salts may be useful in the production of the compounds of the invention.

A suitable pharmaceutically acceptable salt of the compounds of the invention is, for example, an acid-addition salt, for example an inorganic or organic acid. In addition, a suitable pharmaceutically acceptable salt of the compounds of the invention is an alkali metal salt, an alkaline earth metal salt or a salt with an organic base.

Other pharmaceutically acceptable salts and methods of preparing these salts may be found in, for example, Remington's Pharmaceutical Sciences (18^{th} Edition, Mack Publishing Co.).

Some compounds of the invention may have chiral centres and/or geometric isomeric centres (E- and Z-isomers), and it is to be understood that the invention encompasses all such optical, diastereoisomeric and geometric isomers.

The invention also relates to any and all tautomeric forms of the compounds of the invention.

Methods of Preparation

Some compounds of the present invention may be prepared according to the methods described in PCT/SE2004/000738.

Another aspect of the present invention provides processes for preparing compounds of formula I, or salts, solvates or solvated salts thereof.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting is groups as well as examples of suitable protecting groups are described, for example, in “Protective Groups in Organic Synthesis”, T. W. Green, P. G. M. Wuts, Wiley-Interscience, New York, (1999). References and descriptions of other suitable reactions are described in textbooks of organic chemistry, for example, “Advanced Organic Chemistry”, March, 4^{th} ed. McGraw Hill (1992) or, “Organic Synthesis”, Smith, McGraw Hill, (1994). For representative examples of heterocyclic chemistry see for example “Heterocyclic Chemistry”, J. A. Joule, K. Mills, G. F. Smith, 3^{rd} ed. Chapman and Hall (1995), p. 189-224 and “Heterocyclic Chemistry”, T. L. Gilchrist, 2^{nd} ed. Longman Scientific and Technical (1992), p. 248-282.

The term “room temperature” and “ambient temperature” shall mean, unless otherwise specified, a temperature between 16 and 25° C.

Methods of Preparation

One embodiment of the invention relates to processes for the preparation of the compound of formula I according to Methods A and B, wherein R¹ to R⁹, unless otherwise specified, are defined as in formula I, comprising;
Method A 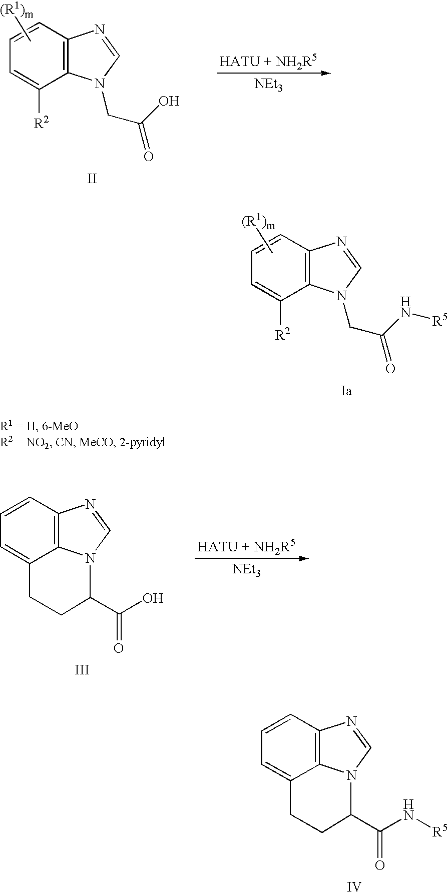
whereby the target compound of formula Ia is obtained from the acid of formula II or its deprotonated form, via its conversion into an activated form, i.e. either the acyl chloride by treatment with oxalyl chloride or the mixed anhydride by treatment with O-(7-azabenzotriazol-1-yl)-N,N,N′,N′-tetramethyluronium hexafluorophosphate and further treatment with an appropriate amine NH₂R⁵. This reaction may be performed in any manner known to the skilled man in the art. The activation may be performed using any other similar activating reagent like 1,3-dicyclohexylcarbodiimide, 1-ethyl-3-(3-dimethylaminopropyl)carbodiimide hydrochloride or 1,1′-carbonyldiimidazole. Suitable solvents to be used for this reaction may be halogenated hydrocarbons such as chloroform, dichloromethane and dichloroethane or aromatic and heteroaromatic compounds such as benzene, toluene, xylene, pyridine and lutidine or ethers such as ethyl ether, tetrahydrofuran and dioxan or aprotic polar solvents like acetonitrile and dimethylformamide, or any mixtures thereof. Catalysts such as heteroaromatic bases like pyridine and lutidine or tertiary amines like triethylamine, N-methylmorpholine and ethyl diisopropylamine may be used as well. The temperature may be between −30 and 50° C. and the reaction time between 1 and 30 h. Starting materials, the acids of formula II, may be obtained using multistep procedures described in detail in the following examples of synthesis, starting from commercially available appropriately 1,2,3-trisubstituted benzenes.
Or,
Method B
whereby the target compound of formula I is obtained from another compound of formula I by a chemical modification of the R² substituent using standard methods described in the literature, for example: 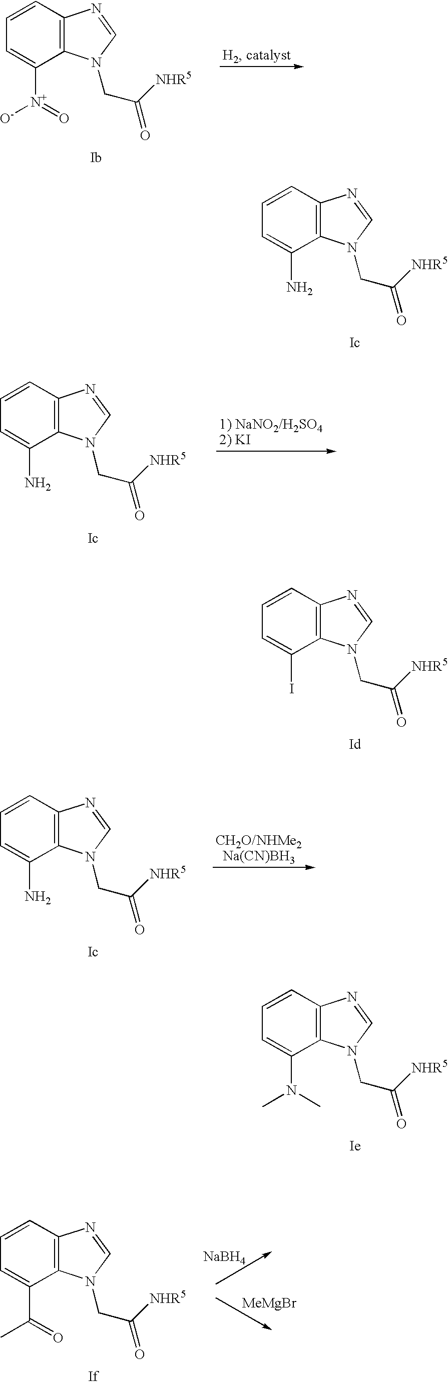 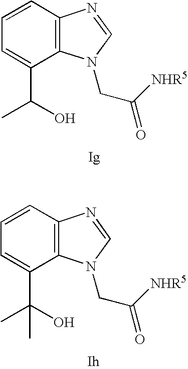
wherein, the target compound of formula I is obtained from an amidoalkylbromide and an appropriately substituted benzimidazole. The amidoalkylbromides mentioned may be obtained by amination of the corresponding carboxyalkyl bromides or their acyl chloride derivatives.

Generally, this method yields a mixture of two regio-isomers, which can be separated by use of chromatography. Suitable solvents to be used for this reaction may be tertiary amides such as dimethylformamide or dimethylacetamide or aromatic compounds such as benzene, toluene and xylene, or ethers such as ethyl ether, tetrahydrofuran and dioxan or alcohols such as methanol, ethanol and propanol, or any mixtures thereof. Bases such as potassium tert-butoxide, sodium methoxide and sodium hydride or tertiary amines like triethylamine, N-methylmorpholine and ethyl diisopropylamine may be used as well. The temperature may be between 0 and 100° C. and the reaction time between 1 and 30 h.

Intermediates

A further embodiment of the invention relates to compounds selected from the group consisting of
3-methoxy-5-(methoxymethyl)aniline,
3-(methoxymethyl)-5-(trifluoromethyl)aniline,
1-(methoxymethyl)-3-nitro-5-(trifluoromethyl)benzene,
1-[3-amino-5-(trifluoromethyl)phenyl]ethanone,
(7-chloro-6-methoxy-1H-benzimidazol-1-yl)acetic acid,
2-[(2-chloro-3-methoxy-6-nitrophenyl)amino]ethanol,
2-(7-chloro-6-methoxy-1H-benzimidazol-1-yl)ethanol,
3-(2-methoxyethoxy)-5-(trifluoromethyl)aniline,
1-(2-methoxyethoxy)-3-nitro-5-(trifluoromethyl)benzene,
3-methoxy-5-(tetrahydrofuran-2-ylmethoxy)aniline,
2-[(3-methoxy-5-nitrophenoxy)methyl]tetrahydrofuran,
3-methoxy-5-(tetrahydrofuran-3-yloxy)aniline,
3-(3-methoxy-5-nitrophenoxy)tetrahydrofuran,
5,6-dihydro-4H-imidazo[4,5,1-ij]quinoline-4-carboxylic acid,
methyl 8-amino-1,2,3,4-tetrahydroquinoline-2-carboxylate,
(7-pyridin-2-yl-1H-benzimidazol-1-yl)acetic acid,
methyl (7-bromo-1H-benzimidazol-1-yl)acetate,
methyl (7-pyridin-2-yl-1H-benzimidazol-1-yl)acetate,
3-methoxy-5-(tetrahydro-2H-pyran-2-ylmethoxy)aniline, and
3-(2-isopropoxyethoxy)-5-methoxyaniline

Another embodiment relates to the use of these compounds as intermediates in the preparation of compounds of the invention.

Pharmaceutical Composition

According to one embodiment of the present invention there is provided a pharmaceutical composition comprising as active ingredient a therapeutically effective amount of the compound of the invention, or salts, solvates or solvated salts thereof, in association with one or more pharmaceutically acceptable diluents, excipients and/or inert carriers.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream or for rectal administration e.g. as a suppository.

In general the above compositions may be prepared in a conventional manner using one or more conventional excipients, pharmaceutical acceptable diluents and/or inert carriers. Suitable daily doses of the compounds of the invention in the treatment of a mammal, including man, are approximately 0.01 to 250 mg/kg bodyweight at peroral administration and about 0.001 to 250 mg/kg bodyweight at parenteral administration.

The typical daily dose of the active ingredient varies within a wide range and will depend on various factors such as the relevant indication, severity of the illness being treated, the route of administration, the age, weight and sex of the patient and the particular compound being used, and may be determined by a physician.

Examples of Pharmaceutical Composition

The following illustrate representative pharmaceutical dosage forms containing a compound of the invention, or salts, solvates or solvated salts thereof, (hereafter compound X), for preventive or therapeutic use in mammals:

| (a): Tablet | mg/tablet |
|---|---|
| Compound X | 100 |
| Lactose | 182.75 |
| Croscarmellose sodium | 12.0 |
| Maize starch paste (5% w/v paste) | 2.25 |
| Magnesium stearate | 3.0 |

| (b): Capsule | mg/capsule |
|---|---|
| Compound X | 10 |
| Lactose | 488.5 |
| Magnesium stearate | 1.5 |

| (c): Injection | (50 mg/ml) |
|---|---|
| Compound X | 5.0% w/v |
| 1M Sodium Hydroxide solution | 15.0% v/v |
| 0.1M Hydrochloric acid | (to adjust pH to 7.6) |
| Polyethylene glycol 400 | 4.5% w/v |
| Water for injection | up to 100% |

The above compositions may be obtained by conventional procedures well known in the pharmaceutical art.

Medical Use

Surprisingly, it has been found that the compounds according to the present invention are useful in therapy. The compounds of the invention, or salts, solvates or solvated salts thereof, as well as their corresponding active metabolites, exhibit a high degree of potency and selectivity for individual vanilloid receptor 1 (VR1) groups. Accordingly, the compounds of the present invention are expected to be useful in the treatment of conditions associated with excitatory activation of vanilloid receptor 1 (VR1).

The compounds may be used to produce an inhibitory effect of VR1 in mammals, including man.

VR1 are highly expressed in the peripheral nervous system and in other tissues. Thus, it is expected that the compounds of the invention are well suited for the treatment of VR1 mediated disorders.

The compounds of the invention are expected to be suitable for the treatment of acute and chronic pain, acute and chronic neuropathic pain and acute and chronic inflammatory pain.

Examples of such disorder may be selected from the group comprising low back pain, post-operative pain, visceral pains like chronic pelvic pain and the like.

Further relevant disorders may be selected from the group comprising cystitis, including interstitial cystitis and pain related thereto, ischeamic, sciatia, diabetic neuropathy, multiple sclerosis, arthritis, fibromyalgia, psoriasis, cancer, emesis, urinary incontinence, hyperactive bladder and HIV neuropathy.

Additional relevant disorders may be selected from the group comprising gastro-esophageal reflux disease (GERD), irritable bowel syndrome (IBS), inflammatory bowel disease (IBD) and pancreatitis.

Other relevant disorders are related to respiratory diseases and may be selected from the group comprising asthma, cough, chronic obstructive lung disease and emphysema, lung fibrosis and interstitial lung disease.

The VR1 inhibitor(s) may be administrated by either an oral or inhaled route. The respiratory disease may be an acute and chronic illness and may be related to infection(s) and/or exposure to environmental pollution and/or irritants.

The compounds of the invention may also be used as antitoxin to treat (over-) exposure to VR1 activators like capsaicin, tear gas, acids or heat. Regarding heat, there is a potential use for VR1 antagonists in (sun-) burn induced pain, or inflammatory pain resulting from burn injuries.

The compounds may further be used for treatment of tolerance to VR1 activators.

One embodiment of the invention relates to the compounds of the invention as hereinbefore defined, for use as a medicament.

Another embodiment of the invention relates to the compounds of the invention as hereinbefore defined, for use as a medicament for treatment of VR1 mediated disorders.

A further embodiment of the invention relates to the compounds of the invention as hereinbefore defined, for use as a medicament for treatment of acute and chronic pain disorders.

Yet another embodiment of the invention relates to the compounds of the invention as hereinbefore defined, for use as a medicament for treatment of acute and chronic neuropathic pain.

Yet a further embodiment of the invention relates to the compounds of the invention as hereinbefore defined, for use as a medicament for treatment of acute and chronic inflammatory pain.

One embodiment of the invention relates to the compounds of the invention as hereinbefore defined, for use as a medicament for treatment of low back pain, post-operative pain and visceral pains like chronic pelvic pain.

Another embodiment of the invention relates to the compounds of the invention as hereinbefore defined, for use as a medicament for treatment of cystitis, including interstitial cystitis and pain related thereto, ischeamic, sciatia, diabetic neuropathy, multiple sclerosis, arthritis, fibromyalgia, psoriasis, cancer, emesis, urinary incontinence, hyperactive bladder and HIV neuropathy.

A further embodiment of the invention relates to the compounds of the invention as hereinbefore defined, for use as a medicament for treatment of gastro-esophageal reflux disease (GERD), irritable bowel syndrome (IBS), inflammatory bowel disease (IBD) and pancreatitis.

Yet a further embodiment of the invention relates to the compounds of the invention as hereinbefore defined, for use as a medicament for treatment of respiratory diseases selected from the group comprising asthma, cough, chronic obstructive lung disease and emphysema, lung fibrosis and interstitial lung disease.

One embodiment of the invention relates to the use of the compound of the invention as hereinbefore defined, in the manufacture of a medicament for treatment of VR1 mediated disorders and for treatment of acute and chronic pain disorders, acute and chronic neuropathic pain and acute and chronic inflammatory pain, and respiratory diseases and any other disorder mentioned above.

Another embodiment of the invention relates to a method of treatment of VR1 mediated disorders and acute and chronic pain disorders, acute and chronic neuropathic pain and acute and chronic inflammatory pain, and respiratory diseases, and any other disorder mentioned above, comprising administering to a mammal, including man in need of such treatment, a therapeutically effective amount of the compounds of the invention, as hereinbefore defined.

A further embodiment of the invention relates to a pharmaceutical composition comprising a compound of the invention as hereinbefore defined, for use in treatment of VR1 mediated disorders and for treatment of acute and chronic pain disorders, acute and chronic neuropathic pain and acute and chronic inflammatory pain, and respiratory diseases, and any other disorder mentioned above.

In the context of the present specification, the term “therapy” and “treatment” includes prevention and prophylaxis, unless there are specific indications to the contrary. The terms “treat”, “therapeutic” and “therapeutically” should be construed accordingly.

In this specification, unless stated otherwise, the term “inhibitor” and “antagonist” mean a compound that by any means, partly or completely, blocks the transduction pathway leading to the production of a response by the ligand.

The term “disorder”, unless stated otherwise, means any condition and disease associated with vanilloid receptor activity.

Non-Medical Use

In addition to their use in therapeutic medicine, the compounds of the invention, or salts, solvates or solvated salts thereof, are also useful as pharmacological tools in the development and standardisation of in vitro and in vivo test systems for the evaluation of the effects of inhibitors of VR1 related activity in laboratory animals such as cats, dogs, rabbits, monkeys, rats and nice, as part of the search for new therapeutics agents.

### EXAMPLES

The invention will now be illustrated by the following non-limiting examples.

Abbreviations

DCE dichloroethane
DCM dichloromethane
DMAP dimethylaminopyridine
EDC 1-(3-dimethylaminopropyl)-3-ethylcarbodiimide hydrochloride
HATU O-(7-azabenzotriazol-1-yl)-N,N,N′,N′-tetramethyluronium hexafluorophosphate
HPLC high performance liquid chromatography
LC liquid chromatography
MS mass spectometry
ret. time retention time
TFA trifluoroacetic acid
THF tetrahydrofurane
DMF dimethylformamide
TMEDA tetramethylethylenediamine
EtOAc ethyl acetate
   General Methods

All starting materials are commercially available or described in the literature. The ¹H NMR spectra were recorded on Brucker at 400 MHz. The mass spectra were recorded utilising electrospray (LC-MS; LC:Waters 2790, column XTerra MS C₈ 2.5 μm 2.1×30 mm, buffer gradient H₂O+0.1% TFA:CH₃CN+0.04% TFA, MS: micromass ZMD//ammonium acetate buffer) ionisation techniques.

### Synthesis of the Intermediates

### 7-substituted 1H-benzimidazol-1-yl-acetic acids, 1) thru 7)

### 1) (7-Nitro-1H-benzimidazol-1-yl)acetic acid (triethylammonium salt)

A. (7-Nitro-1H-benzimidazol-1-yl)acetonitrile

A solution (1 M) of potassium tert-butoxide (16.1 ml)) was slowly added to a solution of 4(7)-nitro-1H-benzoimidazole (2.50 g, 15.3 mmol) in dry DMF (100 ml) at 0-5° C. and the resulting dark-red solution was stirred for 15 min at room temperature. Bromoacetonitrile (1.12 mL, 16.1 mmol) was added in one portion and the reaction mixture was stirred for an additional hour, then quenched with dry ice and poured into 400 mL of cold water. The resulting clear solution was repeatedly extracted with CHCl₃ (4×80 ml). Organic extracts were pooled and washed with water (3×50 ml) and brine, dried over Na₂SO₄ and concentrated, yielding a 1:1 mixture of (4-nitro-1H-benzoimidazol-1-yl)acetonitrile and (7-nitro-1H-benzoimidazol-1-yl)acetonitrile. The regioisomers were separated on preparative HPLC (XTerra C₈ column 19×300 mm, 0.1 M aqueous NH₄Ac/CH₃CN), to yield (7-nitro-1H-benzoimidazol-1-yl)acetonitrile, 1.15 g (37%). MS (ESI) m/z: 203.05 [M+H]. ¹H NMR (400 MHz, DMSO-D6) δ ppm 5.68 (s, 2H) 7.50 (t, J=7.8 Hz, 1H) 8.16 (m, 1H) 8.18 (dd, J=8.1, 1.0 Hz, 1H) 8.57 (s, 1H).

B. (7-Nitro-1H-benzoimidazol-1-yl)acetonitrile (1.1 g, 5.4 mmol) was dissolved in 18% hydrochloric acid (30 ml), the solution was transferred into a vial, which was sealed and heated at 105° C. for 6 h. The vial was cooled, the volatiles were removed under reduced pressure and the residue was co-evaporated two times with acetonitrile. To the residue were added dichloromethane (15 ml) and triethylamine (1 ml), and the slurry was purified on a silica gel column using a mixture of dichloromethane/methanol/triethylamine 84:15:1 (v/v/v) as an eluent to yield the title compound, 1.2 g (69%). MS (ESI) m/z: 221.98 [MEt₃N+H]. ¹H NMR (400 MHz, DMSO-D6) δ ppm 1.14 (t, J=7.1 Hz, 9H) 2.97 (q, J=7.1 Hz, 6H) 5.01 (s, 2H) 7.36 (t, J=8.1 Hz, 1H) 7.93 (dd, J=8.1, 1.0 Hz, 1H) 8.06 (m, 1H) 8.37 (s, 1H).

### 2) [7-(ethoxycarbonyl)-1H-benzimidazol-1-yl]acetic acid

A. 2-[(2-Hydroxyethyl)amino]-3-nitrobenzoic acid

2-Chloro-3-nitrobenzoic acid (5.0 g, 24.8 mmol) was suspended in ethanol (90 ml) and ethanolamine (4.5 mL, 74.8 mmol) was added. The resulting clear solution was heated at 100° C. for two days. The volatiles were removed under reduced pressure. The residue was treated with water (40 ml) and the mixture was acidified with 1M hydrochloric acid to pH 2. A yellow precipitate formed was collected by filtration and washed with water to yield 2-(2-hydroxyethylamino)-3-nitrobenzoic acid, 5.14 g (92%). MS (ESI) m/z 225 [M−H]. ¹H NMR (400 MHz, CD₃OD) δ ppm 3.04 (t, J=5.31 Hz, 2H), 3.69 (t, J=5.31 Hz, 2H), 6.71 (t, J=7.96 Hz, 1H), 7.93 (dd, J=8.21, 1.64 Hz, 1H), 8.13 (dd, J=7.71, 1.64 Hz, 1H).

B. Methyl 2-[(2-hydroxyethyl)amino]-3-nitrobenzoate

2-(2-Hydroxyethylamino)-3-nitrobenzoic acid (5.14 g, 22.7 mmol) was dissolved in methanol (200 ml) and concentrated H₂SO₄ (10 ml) was added. The mixture was heated at reflux for 2.5 h. The solvent was removed at reduced pressure. The residue was treated with water (100 ml) and extracted with ethyl acetate (3×150 ml). The combined organic phase was dried and concentrated. Purification by column chromatography on silica using heptane ethyl acetate 1:1 as an eluent afforded methyl 2-[(2-hydroxyethyl)amino]-3-nitrobenzoate, 3.92 g (72%). MS (ESI) m/z 241 [M+H]. ¹H NMR (400 MHz, CDCl₃) δ ppm 3.12 (t, J=5.10 Hz, 2H), 3.84 (t, J=5.15 Hz, 2H), 3.91 (s, 3H), 6.69 (t, J=7.96 Hz, 1H), 7.95 (dd, J=8.34, 1.52 Hz, 1H), 8.08 (dd, J=7.83, 1.52 Hz, 1H).

C. Methyl 1-(2-hydroxyethyl)-1H-benzimidazole-7-carboxylate

Suspension of methyl 2-[(2-hydroxyethyl)amino]-3-nitrobenzoate (3.06 g, 12.7 mmol) in methanol (130 ml) was hydrogenated at atmospheric pressure over 10% palladium on activated charcoal for 10 min. The mixture was filtered through a pad of Celite and the solvent was removed in vacuum. The residue was dissolved in formic acid (60 ml) and heated at 100° C. for 45 min and then kept at ambient temperature overnight. Excess of the formic acid was removed under reduced pressure. The residue was dissolved in methanol (100 ml) and treated with concentrated ammonia in methanol (20 ml) for 50 min followed by evaporation of the volatiles. Purification by column chromatography on silica using dichloromethane in methanol 95:5 afforded methyl 1-(2-hydroxyethyl)-1H-benzimidazole-7-carboxylate, 2.31 g (83%). %). MS (ESI) m/z 221 [M+H]. ¹H NMR (400 MHz, CD₃OD) δ ppm 3.78 (t, J=5.05 Hz, 2H), 3.96 (s, 3H), 4.70 (t, J=5.05 Hz, 2H), 7.33 (t, J=7.83 Hz, 1H), 7.84-7.91 (m, 2H), 8.20 (s, 1H).

D. To a solution of methyl 1-(2-hydroxyethyl)-1H-benzimidazole-7-carboxylate (2.83 g, 12.8 mmol) in acetone (140 ml) a solution of CrO₃ (1.77 g, 17.7 mmol) and concentrated H₂SO₄ (1.77 ml) in water (5 ml) was added. The resulting yellow solution was stirred at ambient temperature for 1 h, while the mixture had changed colour to blue green, and then was quenched by the addition of isopropanol. The volatiles were removed in vacuum. The residue was treated with brine and pH of the solution was adjusted to 3 by addition of aqueous sodium bicarbonate. The water phase was repeatedly extracted with ethyl acetate containing 5% methanol. Drying of the organic phase with sodium sulfate, evaporation of solvent and purification of the residue by column chromatography on silica using a gradient of 10-25% methanol in dichloromethane afforded the title compound, 1.44 g (48%). MS (ESI) m/z 235 [M+H]. ¹H NMR (400 MHz, D₂O) δ ppm 3.95 (s, 3H), 5.17 (s, 2H), 7.57 (t, J=7.95 Hz, 1H), 7.96-8.05 (m, 2H), 8.79 (s, 1H).

### 3) (7-Cyano-1H-benzimidazol-1-yl)acetic acid

A. 2-[(2-Hydroxyethyl)amino]-3-nitrobenzonitrile

A solution of 2-chloro-3-nitrobenzonitrile [prepared as described in WO 97/38983] (0.26 g, 1.4 mmol) and ethanolamine (0.22 mL, 3.5 mmol) in dry ethanol (3.8 ml) was irradiated in a microwave oven at 135° C. for 180 min. The reaction mixture was concentrated under reduced pressure. The residue was dissolved in ethyl acetate, the organic phase was washed with potassium bisulfate (0.1 M), water and brine, dried over Na₂SO₄ and concentrated. Purification was performed using flash chromatography on a silica column and 25% ethyl acetate in heptane as an eluent to yield 2-[(2-hydroxyethyl)amino]-3-nitrobenzonitrile, 0.28 g (95%). ¹H NMR (400 MHz, CD₃CN) δ ppm: 3.00 (t, J=4.8 Hz, 1H), 3.68 (q, J=4.7 Hz, 2H), 3.81 (m, 2H), 6.70 (dd, J=8.6, 7.6 Hz, 1H), 7.75 (dd, J=7.6, 1.5 Hz, 1H), 8.28 (dd, J=8.6, 2.0 Hz, 1H), 8.41 (bs, 1H).

B. 3-Amino-2-[(2-hydroxyethyl)amino]benzonitrile

To a solution of 2-[(2-hydroxyethyl)amino]-3-nitrobenzonitrile (1.55 g, 7.5 mmol) in a mixture of methanol (30 ml) and water (15 ml) sodium acetate trihydrate (56 g) was added. To this mixture titanium trichloride (65 mL, as 15% solution in 10% aqueous HCl) was added drop-wise over period of 20 min. The resulting dark solution was allowed to stir for additional 2 h, and then carefully neutralized with saturated aqueous sodium bicarbonate. The solids were filtered off, and washed with ethyl acetate. The combined organic phase was washed with water and brine, dried over Na₂SO₄ and concentrated yielding 3-amino-2-[(2-hydroxyethyl)amino]benzonitrile (1.23 g, 93%) that was used in the next step without further purification. MS (ESI) m/z: 178 [M+H].

C. 1-(2-Hydroxyethyl)-1H-benzimidazole-7-carbonitrile

3-Amino-2-[(2-hydroxyethyl)amino]benzonitrile (1 g, 5.4 mmol) was dissolved in formic acid (3 ml) and irradiated in microwave oven at 135° C. for 2 h. The mixture was cooled and treated with 37% hydrochloric acid (1 ml) at 50° C. for 0.5 h. The volatiles were removed under reduced pressure. The residue was partitioned between ethyl acetate and saturated aqueous sodium bicarbonate. The organic phase was washed with water and brine, dried over sodium sulfate and concentrated to yield 1-(2-hydroxyethyl)-1H-benzimidazole-7-carbonitrile, 0.9 g (90%). MS (ESI) m/z 188.1 [M+H]. ¹H NMR (400 MHz, DMSO-D6) δ ppm: 3.81 (q, J=5.1 Hz, 2H), 4.53 (t, J=5.3 Hz, 2H), 5.03 (t, J=5.1 Hz, 1H) 7.36 (t, J=7.8 Hz, 1H), 7.76 (dd, J=7.6, 1.0 Hz, 1H), 8.04 (dd, J=8.1, 1.0 Hz, 1H), 8.37 (s, 1H).

D. To a solution of 1-(2-hydroxyethyl)-1H-benzimidazole-7-carbonitrile (0.86 g, 4.6 mmol) in acetone (150 ml) Jones reagent (a mixture of CrO₃ 0.5 g, 5 mmol; H₂SO₄ 0.5 mL in a minimal amount of water to form a clear solution) was added. The reaction mixture was stirred for 6 h, quenched with 2-propanol (2 ml) and concentrated to a quarter of the initial volume. The residue was partitioned between ethyl acetate and aqueous potassium hydrosulfate (0.1 M). The aqueous phase was extracted 3-4 times with ethyl acetate and the combined organic extract was washed with brine, dried over Na₂SO₄ and concentrated. The oily residue was dissolved in a mixture of dichloromethane (15 ml) and triethylamine (2 ml) and the resulting slurry was loaded onto a flash silica column and eluted with a mixture of dichloromethane/methanol/triethylamine 84:15:1. Fractions containing product were pooled, diluted with dioxane (20 ml), evaporated to dryness and dried in vacuo at 40° C. to yield the title product: (7-Cyano-1H-benzimidazol-1-yl)acetic acid, 0.36 g (39%). MS (ESI) m/z 202.0 [M+H]. ¹H NMR (400 MHz, DMSO-D6) δ ppm: 5.31 (s, 2H), 7.37 (dd, J=8.1, 7.7 Hz, 1H), 7.75 (dd, J=7.6, 0.8 Hz, 1H), 8.04 (dd, J=8.1, 1.1 Hz, 1H), 8.38 (s, 1H), 13.43 (bs, 1H).

### 4) (7-Acetyl-1H-benzimidazol-1-yl)acetic acid

A. 1-[1-(2-Hydroxyethyl)-1H-benzimidazol-7-yl]ethanone.

A solution of 1-(2-hydroxyethyl)-1H-benzimidazole-7-carbonitrile (0.29 g, 1.5 mmol) in dry THF (6.2 ml) was cooled to −78° C. and MeLi (5.8 mL, 9.3 mmol) was added slowly. After the addition the reaction mixture was allowed to warm up to ambient temperature and kept such for 30 min. Then the temperature was brought down to −78° C. again and water (4 ml) was added slowly. After warming up the reaction mixture was acidified to pH 4 and heated at 50° C. for 30 min. Solvents were removed under reduced pressure and the residue was partitioned between ethyl acetate and aq. NaHCO₃. The organic extract was farther washed with water and brine, dried over Na₂SO₄ and concentrated. Purification was performed on flash silica column using ethyl acetate-methanol as the eluent.

Yield 0.25 g (80%). Calculated for C₁₁H₁₂N₂O₃ m/z: 204.23, found 205.23 [M+H]⁺. ¹H NMR (400 MHz, DMSO-D6) δ ppm 2.67 (s, 3H) 3.51 (q, J=5.1 Hz, 2H) 4.41 (t, J=5.3 Hz, 2H) 4.77 (t, J=5.1 Hz, 1H) 7.29 (t, J=7.8 Hz, 1H) 7.78 (dd, J=7.6, 1.0 Hz, 1H) 7.88 (dd, J=8.1, 1.0 Hz, 1H) 8.20 (s, 1H).

B. The title compound: (7-acetyl-1H-benzimidazol-1-yl)acetic acid, was prepared and isolated as a triethylammonium salt according to the procedure described for the synthesis of (7-Cyano-1H-benzimidazol-1-yl)acetic acid (part D). Yield 116 mg (30%). Calculated for C₁₁H₁₀N₂O₃ m/z: 218.21, found 219.16 [M+H]⁺. ¹H NMR (400 MHz, DMSO-D6) δ ppm 1.02 (t, J=7.1 Hz, 9H) 2.56 (s, 3H) 2.68-2.77 (m, 6H) 4.91 (s, 2H) 7.24 (t, J=7.8 Hz, 1H) 7.70 (d, J=7.6 Hz, 1H) 7.81-7.85 (m, 1H) 8.16 (s, 1H).

### 5) (7-Pyridin-2-yl-1H-benzimidazol-1-yl)acetic acid

A. Methyl (7-bromo-1H-benzimidazol-1-yl)acetate

To a solution of (7-bromo-1H-benzimidazol-1-yl)acetic acid triethylamine salt (0.42 g, 1.2 mmol) in methanol (20 ml), conc. H₂SO₄ (2.3 ml) was added and the resulting mixture was heated under reflux for 2 h. After cooling the mixture was concentrated to 1/4 of original volume and partitioned between ethyl acetate and aq. NaHCO₃. The organic extract was further washed with water and brine, dried over Na₂SO₄ and concentrated.

Yield 0.38 g (97%). Calculated for C₁₀HgBrN₂O₂ m/z: 267.99, found 269.08 [M+H]⁺. ¹H NMR (400 MHz, DMSO-D6) δ ppm 3.72 (s, 3H) 5.42 (s, 2H) 7.15 (t, J=7.8 Hz, 1H) 7.41-7.46 (m, 1H) 7.69 (dd, J=8.1, 1.0 Hz, 1H) 8.25 (s, 1H).

B. Methyl (7-pyridin-2-yl-1H-benzimidazol-1-yl)acetate

To a mixture of methyl (7-bromo-1H-benzimidazol-1-yl)acetate (108 mg, 0.4 mmol), Pd(dppb)Cl₂ (12 mg), copper(II) oxide (32 mg) in DMF (1.6 ml) under argon, 2-(tributylstannyl)pyridine (0.19 mL, 0.48 mmol) in DMF (0.4 ml) was added in one portion. The reaction mixture was heated at 100° C. for 23 h in a sealed vial. The vial was cooled and opened and the contents were filtered and concentrated. Purification was performed on flash silica column using heptane-ethyl acetate.

Yield 56 mg (52%). Calculated for C₁₅H₁₃N₃O₂ m/z: 267.10, found 268.12 [M+H]⁺. ¹H NMR (400 MHz, MeOD) δ ppm 3.42 (s, 3H) 5.02 (s, 2H) 7.33 (dd, J=7.3, 1.3 Hz, 1H) 7.39 (t, J=7.8 Hz, 1H) 7.43-7.50 (m, 1H) 7.59-7.66 (m, 1H) 7.79 (dd, J=7.8, 1.3 Hz, 1H) 7.91-7.99 (m, 1H) 8.16 (s, 1H) 8.58-8.65 (m, 1H).

C. (7-Pyridin-2-yl-1H-benzimidazol-1-yl)acetic acid triethylamine salt.

Methyl (7-pyridin-2-yl-1H-benzimidazol-1-yl)acetate (50 mg, 0.19 mmol) was dissolved in 3 mL methanol and 2 M aq. NaOH (3 ml) was added. The resulting solution was heated at 45° C. until the completion of hydrolysis (3 h) and then concentrated to dryness. The residue was acidified with 5 M aq. HCl, concentrated to dryness, then redissolved in a mixture of dichloromethane (5 ml) and triethylamine (0.7 ml) and the resulting slurry was loaded onto a flash silica column and eluted with a mixture of dichloromethane/methanol/triethylamine 84:15:1. Fractions containing product were pooled, diluted with dioxane (10 ml), evaporated to dryness and dried under vacuum at 40° C. to yield the title product, 31 mg (47%).

Calculated for C₁₄H₁₁N₃O₂ m/z: 253.09, found 254.14 [M+H]⁺.

### 6) 5,6-Dihydro-4H-imidazo[4,5,1-ij]quinoline-4-carboxylic acid Hydrochloride

A. Methyl 8-amino-1,2,3,4-tetrahydroquinoline-2-carboxylate

Palladium on carbon (10%, 54 mg) was added to a solution of methyl 4-chloro-8-nitroquinoline-2-carboxylate (127 mg, 0.476 mmol) in ethyl acetate (8 ml) and methanol (8 ml), and the mixture was hydrogenated at 1 atmosphere for 40 min. The catalyst was filtered off, and platinum(IV) oxide (56 mg) was added to the filtrate. The mixture was hydrogenated over 3 h at 1 atmosphere. The catalyst was filtered off, and the filtrate was concentrated in vacuo. The residue was purified by column chromatography on silica using heptane/ethyl acetate, 60:40, as the eluent affording 28 mg (29% yield) of the title compound as a yellow oil. MS (ESI) m/z 207 [M+H]. ¹H NMR (400 MHz, DMSO-D6) δ ppm 1.95-2.00 (m, 2H), 2.50-2.54 (m, 1H, partly overlapped with the DMSO peak), 2.60-2.67 (m, 1H), 3.66 (s, 3H), 4.08-4.11 (m, 1H), 4.39 (s, 2H), 4.82 (d, J=2.8 Hz, 1H), 6.22 (m, 1H), 6.33 (t, J=7.4 Hz, 1H), 6.38-6.40 (m, 1H).

B. A solution of methyl 8-amino-1,2,3,4-tetrahydroquinoline-2-carboxylate (28 mg, 0.136 mmol) in formic acid (3 ml) was heated at 100° C. for 1 h. The excess of solvent was removed in vacuo, and the residual oil was dissolved in a 6 M hydrochloric acid solution and heated at reflux for 30 min. The solvent was removed in vacuo affording 32 mg (100% yield) of the title compound as a pink solid. MS (ESI) m/z 203 [M−HCl+H]. ¹H NMR (400 MHz, DMSO-D6) δ ppm 2.36-2.46 (m, 1H), 2.61-2.66 (m, 1H), 2.80-2.88 (m, 1H), 3.10-3.16 (m, 1H), 5.66 (t, J=4.2 Hz, 1H), 7.41 (d, J=7.3 Hz, 1H), 7.53 (t, J=7.8 Hz, 1H), 7.70 (d, J=8.3 Hz, 1H), 9.63 (s, 1H).

### 7) (7-Chloro-6-methoxy-1H-benzimidazol-1-yl)acetic acid

A. 2-[(2-Chloro-3-methoxy-6-nitrophenyl)amino]ethanol

A solution of 2,3-dichloro-1-methoxy-4-nitrobenzene (225 mg, 1.01 mmol) and ethanolamine (309 mg, 5.07 mmol) in ethanol (4 ml) was heated at reflux overnight. Additional ethanolamine (500 mg, 8.20 mmol) was added, and the solution was heated for another 8 h. The solvent was removed in vacuo, and the residue was partitioned between water and ethyl acetate. The organic layer was washed with brine, dried over magnesium sulfate, and the solvent was removed in vacuo. The residue was purified by column chromatography on silica using heptane/ethyl acetate, 70:30, as an eluent affording 141 mg (56% yield) of the title compound as an orange solid. MS (ESI) m/z 247 [M+H]. ¹H NMR (400 MHz, DMSO-D6) δ ppm 3.35-3.39 (m, 2H), 3.50-3.54 (m, 2H), 3.95 (s, 3H), 4.85 (t, J=5.0 Hz, 1H), 6.75 (d, J=9.6 Hz, 1H), 7.10 (broad t, J=5.3 Hz, 1H), 8.02 (d, J=9.4 Hz, 1H).

B. 2-(7-Chloro-6-methoxy-1H-benzimidazol-1-yl)ethanol

The title compound was synthesized according to the procedure described for the synthesis of (7-Cyano-1H-benzimidazol-1-yl)acetic acid, part B and C, starting from 2-[(2-chloro-3-methoxy-6-nitrophenyl)amino]ethanol. Yield 93 mg (74%). MS (ESI) m/z 227 [M+H]. ¹H NMR (400 MHz, DMSO-D6) δ ppm 3.72-3.76 (m, 2H), 3.89 (s, 3H), 4.51 (t, J=5.6 Hz, 2H), 4.95 (t, J=5.3 Hz, 1H), 7.10 (d, J=8.8 Hz, 1H), 7.58 (d, J=8.6 Hz, 1H), 8.05 (s, 1H).

C. The title compound was synthesized according to the procedure described for the synthesis of (7-Cyano-1H-benzimidazol-1-yl)acetic acid, part D, starting from 2-(7-chloro-6-methoxy-1H-benzimidazol-1-yl)ethanol. Yield 40 mg (44%). MS (ESI) m/z 241 [M+H]. The material was used as such without further purification in the synthesis of the target compound.

### Syntheses of the Intermediates

### Amines, 8) thru 15)

### 8) 3-Methoxy-5-(methoxymethyl)aniline

1-Methoxy-3-(methoxymethyl)-5-nitrobenzene (197 mg, 1 mmol) dissolved in methanol (5 ml) was hydrogenated over 10% Pd/C at 40 psi for 2 h at ambient temperature. The reaction mixture was filtered through Celite to remove the catalyst. The filtrate was concentrated in vacuum to yield 3-methoxy-5-(methoxymethyl)aniline (154 mg, 92%). ¹H NMR (400 MHz, DMSO-d6) δ ppm 3.23 (s, 3H), 3.64 (s, 3H), 4.24 (s, 2H), 5.01 (br.s, 2H), 6.41 (br.d, J=7.6 Hz, 1H), 6.45 (dd, J=8.1, 2.0 Hz, 1H), 6.51 (t, J=1.7 Hz, 1H), 6.95 (t, J=8.0 Hz, 1H)

### 9) 3-(Methoxymethyl)-5-(trifluoromethyl)aniline

To a stirred solution of [3-nitro-5-(trifluoromethyl)phenyl]methanol (221 mg, 1 mmol) in THF (1 ml) a solution of potassium tert-butoxide (1M, 1.1 ml, 1.1 mmol) in THF was addes at −78° C. followed by an addition of methyl iodide (213 mg, 1.5 mmol). The mixture was allowed to reach ambient temperature and was stirred for additional 2 h. The mixture was quenched with water and extracted with chloroform. The extract was dried over sodium sulphate and concentrated in vacuum. The crude product was purified chromatographically on silica gel using 20% ethyl acetate in heptane as an eluent to yield 1-(methoxymethyl)-3-nitro-5-(trifluoromethyl)benzene (130 mg, 55%). ¹H NMR (400 MHz, CDCl₃) □ ppm 3.48 (s, 3H), 4.6 (s, 2H), 7.93 (s, 1H), 8.38 (br.s, 2H). 1-(Methoxymethyl)-3-nitro-5-(trifluoromethyl)benzene (118 mg, 0.5 mmol) was hydrogenated over 10% Pd/C at 40 psi for 3 h at ambient temperature. The reaction mixture was filtered through Celite to remove the catalyst. The filtrate was concentrated in vacuum to yield 3-(methoxymethyl)-5-(trifluoromethyl)aniline (82 mg, 80%). ¹H NMR (400 MHz, CDCl₃) δ ppm 3.39 (s, 3H), 3.8 (br.s, 2H), 4.39 (s, 2H), 6.80 (br.s, 2H), 6.94 (br.s, 1H)

### 10) 1-[3-Amino-5-(trifluoromethyl)phenyl]ethanone

3-Amino-5-(trifluoromethyl)benzonitrile (186 mg, 1 mmol) dissolved in THF (1 ml) was treated with methyl lithium (1.4M in THF, 2.15 ml, 3 mmol) at −78° C. The mixture was allowed to reach gradually −20° C. and stirred for additional 0.5 h. The mixture was quenched with water, acidified with hydrochloric acid to pH 1-2 and warmed gently to 40-45° C. for 0.5 h. The mixture was neutralised with sodium bicarbonate and extracted with chloroform. The extract was dried over sodium sulphate and concentrated in vacuum. The crude product was purified using preparative HPLC to yield 1-[3-amino-5-(trifluoromethyl)phenyl]ethanone (108 mg, 53%). Calculated for C₉H₈F₃NO m/z: 203.2, found 204.1 [M+H]⁺. ¹H NMR (400 MHz, CDCl₃) δ ppm 2.58 (s, 3H), 3.71 (br.s, 2H), 7.07 (s, 1H), 7.39 (s, 1H), 7.52 (s, 1H)

### 11) 3-Methoxy-5-(tetrahydrofuran-3-yloxy)aniline

A. 3-(3-Methoxy-5-nitrophenoxy)tetrahydrofuran

A solution of diethyl azodicarboxylate (40% solution in toluene, 371 mg, 0.85 mmol) in tetrahydrofuran (0.7 ml) was added to a solution of 3-methoxy-5-nitrophenol (111 mg, 0.66 mmol), triphenylphosphine (310 mg, 1.18 mmol), and 3-hydroxytetrahydrofuran (69 mg, 0.79 mmol) in tetrahydrofuran (2 ml). The reaction mixture was stirred at ambient temperature for 4 h. The solvent was removed in vacuo, and the residue was partitioned between a 1 M solution of sodium hydroxide and ethyl acetate. The organic layer was washed with a 1 M solution of sodium hydroxide followed by a saturated solution of sodium bicarbonate. The organic layer was dried over magnesium sulfate, and the solvent was removed in vacuo. The residue was purified by column chromatography on silica using heptane/ethyl acetate, 90:10-50:50, as an eluent affording 102 mg (65% yield) of the title compound as a pale yellow solid. MS (EI) m/z 239 (M⁺). ¹H NMR (400 MHz, DMSO-D6) δ ppm 1.94-2.01 (m, 1H), 2.21-2.30 (m, 1H), 3.74-3.83 (m, 3H), 3.86 (s, 3H), 3.87-3.91 (m, 1H), 5.18-5.21 (m, 1H), 6.96 (t, J=2.3 Hz, 1H), 7.31 (t, J=2.0 Hz, 1H), 7.34 (t, J=2.2 Hz, 1H).

B. Palladium on carbon (5%, 30 mg) was added to a solution of 3-(3-methoxy-5-nitrophenoxy)tetrahydrofuran (100 mg, 0.418 mmol) in ethanol (5 ml) and ethyl acetate (1 ml), and the mixture was hydrogenated at 1 atmosphere for 1 h. The catalyst was filtered off, and the filtrate was concentrated in vacuo affording 87 mg (100% yield) of 3-methoxy-5-(tetrahydrofuran-3-yloxy)aniline as an oil. MS (ESI) m/z 210 [M+H]. ¹H NMR (400 MHz, DMSO-D6) δ ppm 1.88-1.95 (m, 1H), 2.10-2.19 (m, 1H), 3.62 (s, 3H), 3.70-3.85 (m, 4H), 4.83-4.86 (m, 1H), 5.05 (s, 2H), 5.64 (t, J=2.2 Hz, 1H), 5.72 (t, J=1.9 Hz, 1H), 5.75 (t, J=1.9 Hz, 1H).

### 12) 3-(2-Methoxyethoxy)-5-(trifluoromethyl)aniline

A. 1-(2-Methoxyethoxy)-3-nitro-5-(trifluoromethyl)benzene

The title compound was synthesized according to the procedure described for the synthesis of 3-methoxy-5-(tetrahydrofuran-3-yloxy)aniline, part A, starting from 3-nitro-5-(trifluoromethyl)phenol and methoxyethanol. Yield 98 mg (50%). ¹H NMR (400 MHz, DMSO-D6) δ ppm 3.32 (s, 3H, overlapped with water peak), 3.69-3.72 (m, 2H), 4.35-4.37 (m, 2H), 7.80 (m, 1H), 8.03 (t, J=2.2 Hz, 1H), 8.05 (m, 1H).

B. 3-(2-Methoxyethoxy)-5-(trifluoromethyl)aniline was synthesized according to the procedure described for the synthesis of 3-methoxy-5-(tetrahydrofuran-3-yloxy)aniline, part B, starting from 1-(2-methoxyethoxy)-3-nitro-5-(trifluoromethyl)benzene. Yield 89 mg. MS (ESI) m/z 236 [M+H]. ¹H NMR (400 MHz, DMSO-D6) δ ppm 3.30 (s, 3H), 3.61-3.64 (m, 2H), 4.03-4.05 (m, 2H), 5.56 (s, 2H), 6.31 (s, 1H), 6.35 (s, 1H), 6.45 (s, 1H).

### 13) 3-Methoxy-5-(tetrahydrofuran-2-ylmethoxy)aniline

A. 2-[(3-Methoxy-5-nitrophenoxy)methyl]tetrahydrofuran

The title compound was synthesized according to the procedure described for the synthesis of 3-methoxy-5-(tetrahydrofuran-3-yloxy)aniline, part A, starting from 2-(hydroxymethyl)tetrahydrofuran. Yield 104 mg (63%). MS (EI) m/z 253 (M⁺). ¹H NMR (400 MHz, DMSO-D6) δ ppm 1.64-1.73 (m, 1H), 1.78-2.04 (m, 3H), 3.65-3.71 (m, 1H), 3.76-3.81 (m, 1H), 3.86 (s, 3H), 4.00-4.04 (m, 1H), 4.08-4.12 (m, 1H), 4.14-4.20 (m, 1H), 6.98 (t, J=2.3 Hz, 1H), 7.32-7.35 (m, 2H).

B. 3-Methoxy-5-(tetrahydrofuran-2-ylmethoxy)aniline was synthesized according to the procedure described for the synthesis of 3-methoxy-5-(tetrahydrofuran-3-yloxy)aniline, part B, starting from 2-[(3-methoxy-5-nitrophenoxy)methyl]tetrahydrofuran. Yield 85 mg (97%). MS (ESI) m/z 224 [M+H]. ¹H NMR (400 MHz, DMSO-D6) δ ppm 1.58-1.67 (m, 1H), 1.75-2.01 (m, 3H), 3.62 (s, 3H), 3.63-3.68 (m, 1H), 3.74-3.82 (m, 3H), 4.06-4.12 (m, 1H), 5.03 (broad s, 2H), 5.67 (t, J=2.2 Hz, 1H), 5.74 (d, J=2.4 Hz, 2H).

### 14) 3-methoxy-5-(tetrahydro-2H-pyran-2-ylmethoxy)aniline

Diisopropyl azodicarboxylate (0.19 mL, 0.99 mmol)) was added dropwise to a mixture of tert-butyl (3-hydroxy-5-methoxyphenyl)carbamate (196 mg, 0.82 mmol), triphenylphosphine (259 mg, 0.99 mmol), and tetrahydropyran-2-methanol (124 mg, 1.07 mmol) in tetrahydrofuran (2.5 mL) under nitrogen atmosphere. The reaction mixture was stirred at room temperature overnight. The mixture was partitioned between a 1 M NaOH solution and ethyl acetate. The organic layer was washed with brine, dried (MgSO₄) and evaporated to give a crude product which was purified by column chromatography to give tert-butyl[3-methoxy-5-(tetrahydro-2H-pyran-2-ylmethoxy)phenyl]carbamate. This material was treated with 30% solution of trifluoroacetic acid in chloroform overnight. After removal of the volatiles in vacuum 3-methoxy-5-(tetrahydro-2H-pyran-2-ylmethoxy)aniline (91 mg, 47%) was isolated as an colourless oil. MS (APCI) m/z 238 [M+H]. ¹H NMR (400 MHz, DMSO-D6) δ ppm 1.22-1.32 (m, 1H), 1.43-1.51 (m, 3H), 1.60-1.63 (m, 1H), 1.79-1.83 (m, 1H), 3.32-3.40 (m, 1H), 3.52-3.58 (m, 1H), 3.61 (s, 3H), 3.71-3.79 (m, 2H), 3.86-3.90 (m, 1H), 5.03 (s, 2H), 5.66-5.67 (m, 1H), 5.73-5.74 (m, 2H).′

### 15) 3-(2-isopropoxyethoxy)-5-methoxyaniline

The title compound was synthesized according to the procedure described for the synthesis of 3-methoxy-5-(tetrahydro-2H-pyran-2-ylmethoxy)aniline starting from tert-butyl (3-hydroxy-5-methoxyphenyl)carbamate and 2-isopropyxyethanol. Yield 78 mg (74%) as an oil. MS (APCI) m/z 226 [M+H]. ¹H NMR (400 MHz, DMSO-D6) δ ppm 1.10 (d, J=6.1 Hz, 6H), 3.58-3.64 (m, 6H), 3.90-3.92 (m, 2H), 5.03 (s, 2H), 5.67 (t, J=2.2 Hz, 1H), 5.74-5.75 (m, 2H).

### Synthesis of the Target Compounds

General Method.

To an ice-cooled solution of a 7-substituted (1H-benzimidazol-1-yl)acetic acid, prepared as described above (0.14 mmol), triethylamine (0.80 mL, 0.56 mmol) and an appropriate amine (commercially available or described in the literature or described above, 0.2 mmol) in acetonitrile (2 ml) O-(7-azabenzotriazol-1-yl)-N,N,N′,N′-tetramethyluronium hexafluoro-phosphate (69 mg, 0.18 mmol)) was added. The ice-bath was removed, and the reaction mixture was stirred at ambient temperature for 0.5-3 h. The mixture was quenched with methanol and the volatiles were removed in vacuo. The residue was purified by column chromatography on silica using a solution of 0-10% methanol in ethyl acetate as an eluent affording the title compound. Alternatively, the residue was purified by preparative HPLC on XTerra C₈ column (19×300 mm) using 0.1 M aqueous NH₄OAc/CH₃CN as an eluent.

| | | | MW | |
|---|---|---|---|---|
| | | | found | |
| | | | [M + 1] | |
| Example | | MW | or | |
| number | Name | calcd | [M − 1] | ¹H NMR |
| 1 | N-{3-[2-(Dimethyl- | 383.4 | 384 | (400 MHz, CD₃OD) δ ppm |
| | amino)ethoxy]phenyl}-2- | | | 2.75 (s, 6 H), 3.30 (t, J = 5.1 Hz, |
| | (7-nitro-1H-benzimidazol- | | | 2 H), 4.22 (t, J = 5.1 Hz, 2 H), |
| | 1-yl)acetamide | | | 5.45 (s, 2 H), 6.73 (dd, |
| | | | | J = 7.6, 2.5 Hz, 1 H), 6.99 (dd, |
| | | | | J = 8.1, 2.0 Hz, 1 H), 7.23 (t, J = 8.1 Hz, |
| | | | | 1 H), 7.35 (t, J = 2.4 Hz, |
| | | | | 1 H), 7.43 (t, J = 8.1 Hz, 1 H), |
| | | | | 8.04-8.09 (m, 2 H), |
| | | | | 8.35 (s, 1 H) |
| 2 | N-[3-(Methoxymethyl) | 340.3 | 341.1 | (400 MHz, CD₃OD) δ ppm |
| | phenyl]-2-(7-nitro- | | | 3.35 (s, 3 H), 4.41 (s, 2 H), |
| | 1H-benzimidazol-1- | | | 5.45 (s, 2 H), 7.07 (d, J = 7.6 Hz, |
| | yl)acetamide | | | 1 H), 7.28 (t, J = 7.9 Hz, 1 H), |
| | | | | 7.40-7.51 (m, 3 H), |
| | | | | 8.05-8.09 (m, 2 H), 8.35 (s, 1 H) |
| 3 | N-(1,3-Dihydro-2-benzo- | 338.3 | 339.1 | (400 MHz, CD₃OD) δ ppm |
| | furan-5-yl)-2-(7-nitro-1H- | | | 5.01 (s, 4 H), 5.40 (s, 2 H), |
| | benzimidazol-1- | | | 7.15 (d, J = 8.6 Hz, 1 H), |
| | yl)acetamide | | | 7.29 (dd, J = 8.1, 1.5 Hz, 1 H), |
| | | | | 7.40 (t, J = 8.1 Hz, 1 H), |
| | | | | 7.47 (d, J = 1.5 Hz, 1 H), 8.05 (d, |
| | | | | J = 8.1 Hz, 1 H), 8.06 (d, J = 7.6 Hz, |
| | | | | 1 H), 8.24 (s, 1 H) |
| 4 | N-[3-Methoxy-5-(methoxymethyl) | 370.4 | 371.1 | (400 MHz, DMSO-d6) δ ppm |
| | phenyl]-2-(7- | | | 3.30 (s, 3 H), 3.69 (s, 3 H), |
| | nitro-1H-benzimidazol-1- | | | 4.33 (s, 2 H), 5.38 (s, 2 H), |
| | yl)acetamide | | | 6.58 (s, 1 H), 7.04 (s, 1 H), |
| | | | | 7.10 (t, J = 2.1 Hz, 1 H), |
| | | | | 7.42 (t, J = 7.9 Hz, 1 H), 8.02 (d, |
| | | | | J = 8.1 Hz, 1 H), 8.14 (dd, |
| | | | | J = 8.1, 1.1 Hz, 1 H), 8.44 (s, 1 H), |
| | | | | 10.39 (br.s, 1 H) |
| 5 | N-[3-(Methoxymethyl)-5- | 408.3 | | (400 MHz, CD₃OD) δ ppm |
| | (trifluoromethyl)phenyl]- | | | 3.39 (s, 3 H), 4.48 (s, 2 H), |
| | 2-(7-nitro-1H-benzimidazol- | | | 5.47 (s, 2 H), 7.35 (s, 1 H), |
| | 1-yl)acetamide | | | 7.44 (t, J = 8.1 Hz, 1 H), |
| | | | | 7.71 (s, 1 H), 7.81 (s, 1 H), 8.08 (d, |
| | | | | J = 8.1 Hz, 2 H), 8.35 (s, 1 H) |
| 6 | N-[3-Cyano-5-(trifluoromethyl) | 389.3 | 390.1 | (400 MHz, CD₃OD) δ ppm |
| | phenyl]-2-(7-nitro- | | | 5.48 (s, 2 H), 7.42 (t, J = 8.1 Hz, |
| | 1H-benzimidazol-1- | | | 1 H), 7.75 (s, 1 H), |
| | yl)acetamide | | | 8.07 (d, J = 8.1 Hz, 2 H), 8.12 (d, |
| | | | | J = 7.2 Hz, 2 H), 8.34 (s, 1 H) |
| 7 | N-[3-Acetyl-5-(trifluoromethyl) | 406.3 | 407.1 | (400 MHz, CD₃OD) δ ppm |
| | phenyl]-2-(7-nitro- | | | 2.61 (s, 3 H), 5.51 (s, 2 H), |
| | 1H-benzimidazol-1- | | | 7.46 (t, J = 8.1 Hz, 1 H), |
| | yl)acetamide | | | 7.95 (s, 1 H), 8.08-8.14 (m, 3 H), |
| | | | | 8.34 (s, 1 H), 8.36 (s, 1 H) |
| 8 | N-[3-(1-Methoxyethyl) | 354.4 | 355.1 | (400 MHz, CD₃OD) δ ppm |
| | phenyl]-2-(7-nitro- | | | 1.36 (d, J = 6.6 Hz, 3 H), |
| | 1H-benzimidazol-1- | | | 3.18 (s, 3 H), 4.25 (q, J = 6.6 Hz, 1 H), |
| | yl)acetamide | | | 5.40 (d, 2 H), 7.02 (d, |
| | | | | J = 7.6 Hz, 1 H), 7.25 (t, J = 7.8 Hz, |
| | | | | 1 H), 7.37-7.45 (m, 3 H), |
| | | | | 8.05 (d, J = 8.1 Hz, 1 H), |
| | | | | 8.07 (d, J = 8.1 Hz, 1 H), 8.26 (s, 1 H) |
| 9 | N-[3-(2-Methoxyethoxy) | 370.4 | 371 | (400 MHz, DMSO-D6) δ ppm |
| | phenyl]-2-(7-nitro- | | | 3.29 (s, 3 H), 3.62-3.64 (m, 2 H), |
| | 1H-benzimidazol-1- | | | 4.01-4.03 (m, 2 H), |
| | yl)acetamide | | | 5.40 (s, 2 H), 6.65 (dd, J = 8.2, 1.9 Hz, |
| | | | | 1 H), 7.04 (d, J = 8.1 Hz, |
| | | | | 1 H), 7.19-7.23 (m, 2 H), |
| | | | | 7.43 (t, J = 8.0 Hz, 1 H), 8.03 (d, J = 8.1 Hz, |
| | | | | 1 H), 8.15 (d, J = 7.6 Hz, |
| | | | | 1 H), 8.46 (s, 1 H), |
| | | | | 10.39 (s, 1 H) |
| 10 | N-[3-Methoxy-5-(2-methoxyethoxy) | 400.4 | 399 | (400 MHz, DMSO-D6) δ ppm |
| | phenyl]-2-(7- | | | 3.29 (s, 3 H), 3.61-3.63 (m, 2 H), |
| | nitro-1H-benzimidazol-1- | | | 3.69 (s, 3 H), |
| | yl)acetamide | | | 4.00-4.02 (m, 2 H), 5.38 (s, 2 H), 6.24 (t, |
| | | | | J = 2.2 Hz, 1 H), |
| | | | | 6.73-6.76 (m, 2 H), 7.43 (t, J = 8.1 Hz, 1 H), |
| | | | | 8.03 (dd, 8.1, 0.8 Hz, 1 H), |
| | | | | 8.14 (dd, 8.0, 0.9 Hz, 1 H), |
| | | | | 8.45 (s, 1 H), 10.36 (s, 1 H) |
| 11 | N-[3-(2-Methoxyethoxy)- | 438.4 | 437 | (400 MHz, DMSO-D6) δ ppm |
| | 5-(trifluoromethyl) | | | 3.80 (s, 3 H), 3.64-3.66 (m, 2 H), |
| | phenyl]-2-(7-nitro- | | | 4.13-4.15 (m, 2 H), |
| | 1H-benzimidazol-1- | | | 5.43 (s, 2 H), 6.98 (s, 1 H), 7.39 (s, |
| | yl)acetamide | | | 1 H), 7.44 (t, J = 8.1 Hz, 1 H), |
| | | | | 7.50 (s, 1 H), 8.04 (d, J = 7.8 Hz, |
| | | | | 1 H), 8.15 (d, J = 8.1 Hz, |
| | | | | 1 H), 8.46 (s, 1 H), 10.76 (s, 1 H) |
| 12 | N-[3-Methoxy-5-(tetrahydrofuran- | 426.4 | 427 | (400 MHz, DMSO-D6) δ ppm |
| | 2-ylmethoxy) | | | 1.61-1.68 (m, 1 H), |
| | phenyl]-2-(7-nitro- | | | 1.76-2.01 (m, 3 H), 3.63-3.67 (m, 1 H), |
| | 1H-benzimidazol-1- | | | 3.69 (s, 3 H), 3.74-3.89 (m, 3 H), |
| | yl)acetamide | | | 4.08-4.14 (m, 1 H), |
| | | | | 5.38 (s, 2 H), 6.24 (t, J = 2.3 Hz, 1 H), |
| | | | | 6.72 (t, J = 1.9 Hz, 1 H), |
| | | | | 6.77 (t, J = 1.9 Hz, 1 H), |
| | | | | 7.43 (t, J = 8.1 Hz, 1 H), 8.03 (dd, J = 8.1, |
| | | | | 1.0 Hz, 1 H), 8.14 (dd, J = 8.1, |
| | | | | 1.0 Hz, 1 H), 8.45 (s, 1 H), |
| | | | | 10.35 (s, 1 H) |
| 13 | N-[3-Methoxy-5-(tetrahydrofuran- | 412.4 | 413 | (400 MHz, DMSO-D6) δ ppm |
| | 3-yloxy)phenyl]- | | | 1.90-1.97 (m, 1 H), |
| | 2-(7-nitro-1H-benzimidazol- | | | 2.13-2.22 (m, 1 H), 3.69 (s, 3 H), |
| | 1-yl)acetamide | | | 3.71-3.86 (m, 4 H), 4.91-4.93 (m, 1 H), |
| | | | | 5.38 (m, 2 H), 6.21 (t, J = 2.3 Hz, |
| | | | | 1 H), 6.74 (m, 2 H), |
| | | | | 7.43 (t, J = 8.1 Hz, 1 H), |
| | | | | 8.03 (dd, J = 7.3, 0.8 Hz, 1 H), |
| | | | | 8.14 (dd, J = 8.0, 0.9 Hz, 1 H), |
| | | | | 8.45 (s, 1 H), 10.37 (s, 1 H) |
| 14 | 2-(7-Nitro-1H-benzimidazol- | 350.3 | 351.0 | (CD₃CN) δ ppm 5.31 (s, 2 H) |
| | 1-yl)-N-(3,4,5- | | | 7.32 (td, J = 10.2, 4.29 Hz, 2 H) |
| | trifluorophenyl)acetamide | | | 7.41 (t, J = 8.1 Hz, 1 H) |
| | | | | 8.05 (dd, J = 8.1, 1.0 Hz, 1 H) |
| | | | | 8.08-8.11 (m, 2 H) 8.84 (s, 1 H) |
| 15 | 2-(7-Nitro-1H-benzimidazol- | 400.4 | 401 | (400 MHz, DMSO-D6) δ ppm |
| | 1-yl)-N-(3,4,5-trimethoxybenzyl) | | | 3.62 (s, 3 H), 3.76 (s, 6 H), |
| | acetamide | | | 4.22 (d, J = 5.8 Hz, 2 H), |
| | | | | 5.24 (s, 2 H), 6.57 (s, 2 H), 7.39 (t, |
| | | | | J = 8.0 Hz, 1 H), 7.99 (dd, |
| | | | | J = 8.1, 0.8 Hz, 1 H), 8.11 (dd, |
| | | | | J = 8.1, 1.0 Hz, 1 H), 8.42 (s, 1 H), |
| | | | | 8.72 (t, J = 5.8 Hz, 1 H) |
| 16 | N-(3,4-Difluorobenzyl)-2- | 346.3 | 347 | (400 MHz, CD₃OD) δ ppm |
| | (7-nitro-1H-benzimidazol- | | | 4.34 (s, 2 H), 5.32 (s, 2 H), |
| | 1-yl)acetamide | | | 7.03-7.09 (m, 1 H), |
| | | | | 7.12-7.22 (m, 2 H), 7.44 (t, J = 8.1 Hz, 1 H), |
| | | | | 8.01-8.07 (m, 2 H), |
| | | | | 8.32 (s, 1 H) |
| 17 | N-[2-(4-Methoxy- | 354.4 | 355 | (400 MHz, CD₃OD) δ ppm |
| | phenyl)ethyl]-2-(7-nitro- | | | 2.70 (t, J = 7.3 Hz, 2 H), |
| | 1H-benzimidazol-1- | | | 3.35 (t, J = 7.3 Hz, 2 H), 3.74 (s, 3 H), |
| | yl)acetamide | | | 5.22 (s, 2 H), |
| | | | | 6.79-6.85 (m, 2 H), 7.09-7.15 (m, 2 H), |
| | | | | 7.44 (t, J = 8.1 Hz, 1 H), |
| | | | | 8.03-8.09 (m, 2 H), 8.28 (s, 1 H) |
| 18 | N-[2-(3-Fluorophenyl) | 342.3 | 343 | (400 MHz, CD₃OD) δ ppm |
| | ethyl]-2-(7-nitro- | | | 2.79 (t, J = 7.2 Hz, 2 H), |
| | 1H-benzimidazol-1- | | | 3.39 (t, J = 7.2 Hz, 2 H), 5.25 (s, 2 H), |
| | yl)acetamide | | | 6.87-6.94 (m, 1 H), |
| | | | | 6.96-7.06 (m, 2 H), 7.24-7.31 (m, 1 H), |
| | | | | 7.44 (t, J = 8.1 Hz, 1 H), |
| | | | | 8.03-8.09 (m, 2 H), 8.29 (s, 1 H) |
| 19 | N-[2-(3-Methoxy- | 355 | 354.4 | (400 MHz, DMF-D9) δ ppm |
| | phenyl)ethyl]-2-(7-nitro- | | | 3.54-3.61 (m, 2 H), 3.80 (s, |
| | 1H-benzimidazol-1- | | | 3 H), 5.36 (s, 2 H), |
| | yl)acetamide | | | 6.6.78-6.89 (m, 3 H), 7.23 (t, J = 7.8 Hz, 1 H), |
| | | | | 7.45 (t, J = 8.1 Hz, 1 H), |
| | | | | 8.05 (dd, J = 8.1, 0.8 Hz, 1 H), |
| | | | | 8.12 (dd, J = 7.8, 1.0 Hz, 1 H), |
| | | | | 8.38 (m, 1 H), 8.49 (s, 1 H) |
| 20 | 2-(7-Nitro-1H-benzimidazol- | 392.3 | 393 | (400 MHz, DMF-D9) δ ppm |
| | 1-yl)-N-{2-[3- | | | 5.35 (s, 2 H), 7.45 (t, J = 8.0 Hz, |
| | (trifluoromethyl)phenyl]ethyl} | | | 1 H), 7.58-7.65 (m, 3 H), |
| | acetamide | | | 7.66 (s, 1 H), 8.05 (dd, J = 8.0. |
| | | | | 0.9 Hz, 1 H), 8.12 (dd, J = 8.1, |
| | | | | 1.0 Hz, 1 H) 8.43 (m, 1 H), |
| | | | | 8.48 (s, 1 H) |
| 21 | N-[2-(3,4-Dimethoxy- | 384.4 | 385 | (400 MHz, DMF-D9) δ ppm |
| | phenyl)ethyl]-2-(7-nitro- | | | 2.70 (t, J = 7.2 Hz, 2 H), |
| | 1H-benzimidazol-1- | | | 3.37 (q, J = 6.7 Hz, 2 H), 3.79 (s, 3 H), |
| | yl)acetamide | | | 3.82 (s, 3 H), 5.35 (s, 2 H), |
| | | | | 6.76 (dd, J = 8.2, 1.9 Hz, 1 H), |
| | | | | 6.89 (s, 1 H), 6.91 (t, J = 1.9 Hz, |
| | | | | 1 H) 7.45 (t, J = 8.1 Hz, 1 H), |
| | | | | 8.05 (dd, J = 8.1, 0.8 Hz, 1 H), |
| | | | | 8.12 (dd, J = 8.0, 0.9 Hz, 1 H), |
| | | | | 8.36 (m, 1 H), 8.50 (s, 1 H) |
| 22 | N-[2-(3,5-Dimethoxy- | 384.4 | 385 | (400 MHz, CD₃OD) δ ppm |
| | phenyl)ethyl]-2-(7-nitro- | | | 2.71 (t, J = 7.2 Hz, 2 H), |
| | 1H-benzimidazol-1- | | | 3.39 (t, J = 7.2 Hz, 2 H), 3.74 (s, 6 H), |
| | yl)acetamide | | | 5.25 (s, 2 H), 6.31 (t, |
| | | | | J = 2.3 Hz, 1 H), 6.41 (d, J = 2.2 Hz, |
| | | | | 2 H), 7.44 (t, J = 8.1 Hz, 1 H), |
| | | | | 8.03-8.09 (m, 2 H), |
| | | | | 8.29 (s, 1 H) |
| 23 | N-(2,3-Dihydro-1H-inden- | 336.4 | 337 | (400 MHz, DMSO-D6) δ ppm |
| | 2-yl)-2-(7-nitro-1H-benzimidazol- | | | 2.72-2.82 (m, 2 H), |
| | 1-yl)acetamide | | | 3.10-3.19 (m, 2 H), 4.33-4.43 (m, 1 H), |
| | | | | 5.13 (s, 2 H), 7.12-7.18 (m, 2 H), |
| | | | | 7.20-7.26 (m, 2 H), |
| | | | | 7.40 (t, J = 8.1 Hz, 1 H), 7.98 (dd, |
| | | | | J = 8.0, 0.9 Hz, 1 H), 8.11 (dd, |
| | | | | J = 8.0, 1.0 Hz, 1 H), 8.40 (s, 1 H), |
| | | | | 8.57 (d, J = 7.1 Hz, 1 H) |
| 24 | N-[2-(5-Bromo-2-methoxyphenyl)- | 433.3 | 434 | (400 MHz, DMSO-D6) δ ppm |
| | ethyl]-2-(7-nitro- | | | 2.65 (t, J = 7.1 Hz, 2 H), |
| | 1H-benzimidazol-1- | | | 3.21 (q, J = 6.7 Hz, 2 H), 3.77 (s, 3 H), |
| | yl)acetamide | | | 5.15 (s, 2 H), 6.94 (d, |
| | | | | J = 8.6 Hz, 1 H), 7.29-7.43 (m, |
| | | | | 3 H), 7.99 (dd, J = 8.1, 0.6 Hz, |
| | | | | 1 H), 8.10 (dd, J = 8.1, 1.0 Hz, |
| | | | | 1 H), 8.33 (t, J = 5.7 Hz, 1 H), |
| | | | | 8.39 (s, 1 H) |
| 25 | N-[1-(4-Chlorobenzyl)-2- | 388.8 | 389 | (400 MHz, DMSO-D6) δ ppm |
| | hydroxyethyl]-2-(7-nitro- | | | 2.60-2.68 (m, 1 H), |
| | 1H-benzimidazol-1- | | | 2.82-2.91 (m, 1 H), 3.25-3.31 (m, 1 H), |
| | yl)acetamide | | | 3.35-3.43 (m, 1 H), |
| | | | | 3.76-3.86 (m, 1 H), 4.83 (t, J = 5.4 Hz, 1 H), |
| | | | | 5.12 (s, 2 H), |
| | | | | 7.18-7.23 (m, 2 H), 7.27-7.33 (m, 2 H), |
| | | | | 7.38 (t, J = 8.1 Hz, 1 H), |
| | | | | 7.97 (dd, J = 8.1, 0.8 Hz, 1 H), |
| | | | | 8.09 (dd, J = 8.0, 0.9 Hz, 1 H), |
| | | | | 8.21 (d, J = 8.6 Hz, 1 H), 8.38 (s, 1 H) |
| 26 | N-(2-Hydroxy-2- | 340.3 | 341 | (400 MHz, DMSO-D6) δ ppm |
| | phenylethyl)-2-(7-nitro- | | | 3.08-3.16 (m, 1 H), |
| | 1H-benzimidazol-1- | | | 3.20-3.27 (m, 1 H), 4.54 (t, J = 6.1 Hz, 1 H), |
| | yl)acetamide | | | 5.18 (s, 2 H), 5.47 (s, 1 H), |
| | | | | 7.20-7.27 (m, 1 H), |
| | | | | 7.28-7.35 (m, 4 H), 7.39 (t, J = 8.0 Hz, 1 H), |
| | | | | 7.98 (dd, J = 8.1, 0.8 Hz, 1 H), |
| | | | | 8.10 (dd, J = 8.1, 1.0 Hz, 1 H), |
| | | | | 8.39 (s, 2 H) |
| 27 | N-(4-Methoxy-2- | 376.2 | 377.1 | (DMSO-D6) δ ppm 3.93 (s, 3 H) |
| | naphthyl)-2-(7-nitro-1H- | | | 5.46 (s, 2 H) 7.10 (d, J = 1.5 Hz, |
| | benzimidazol-1- | | | 1 H) 7.36 (m, 1 H) |
| | yl)acetamide | | | 7.45 (m, 2 H) 7.71 (m, 2 H) |
| | | | | 8.03 (m, 2 H) 8.15 (dd, J = 8.1, 1.0 Hz, |
| | | | | 1 H) 8.48 (s, 1 H) |
| | | | | 10.58 (s, 1 H) |
| 28 | 2-(7-Acetyl-1H-benzimidazol- | 405.4 | | (400 MHz, CD₃OD) δ ppm |
| | 1-yl)-N-[3-(methoxy- | | | 2.60 (s, 3 H), 3.39 (s, 3 H), |
| | methyl)-5-(trifluoro- | | | 4.48 (s, 2 H), 5.43 (s, 2 H), |
| | methyl)phenyl]acetamide | | | 7.34 (s, 1 H), 7.39 (t, J = 7.8 Hz, |
| | | | | 1 H), 7.72 (s, 1 H), |
| | | | | 7.85 (s, 1 H), 7.86 (d, J = 7.6 Hz, 1 H), |
| | | | | 7.92 (d, J = 8.1, 1 H), |
| | | | | 8.22 (s, 1 H) |
| 29 | 2-(7-Acetyl-1H-benzimidazol- | 403.4 | 404.1 | (400 MHz, CD₃OD) δ ppm |
| | 1-yl)-N-[3-acetyl-5- | | | 2.61 (s, 3 H), 2.62 (s, 3 H), |
| | (trifluoromethyl)phenyl]acetamide | | | 5.47 (d, 2 H), 7.40 (t, J = 8.1 Hz, |
| | | | | 1 H), 7.87-7.95 (m, 3 H), |
| | | | | 8.17 (s, 1 H), 8.23 (s, 1 H), |
| | | | | 8.36 (s, 1 H) |
| 30 | 2-(7-Acetyl-1H-benzimidazol- | 386.3 | 387.1 | (400 MHz, CD₃OD) δ ppm |
| | 1-yl)-N-[3-cyano-5- | | | 2.61 (s, 3 H), 5.46 (d, 2 H), |
| | (trifluoromethyl)phenyl]acetamide | | | 7.39 (t, J = 7.9 Hz, 1 H), |
| | | | | 7.77 (br.s, 1 H), 7.89 (dd, J = 7.6, |
| | | | | 1.0 Hz, 1 H), 7.93 (dd, J = 8.1, |
| | | | | 1.0 Hz, 1 H), 8.14-8.18 (m, 2 H), |
| | | | | 8.22 (s, 1 H) |
| 31 | 2-(7-Acetyl-1H-benzimidazol- | 363.4 | 364.2 | (DMSO-D6) δ ppm 1.23 (s, 9 H) |
| | 1-yl)-N-(4-tert-butyl- | | | 4.16 (d, J = 5.6 Hz, 2 H) |
| | benzyl)acetamide | | | 5.12 (s, 2 H) 7.12-7.19 (m, |
| | | | | J = 8.1 Hz, 2 H) 7.26 (t, J = 7.8 Hz, |
| | | | | 1 H) 7.28-7.33 (m, 2 H) |
| | | | | 7.72 (dd, J = 7.6, 1.0 Hz, 1 H) |
| | | | | 7.85 (dd, J = 8.1, 1.0 Hz, 1 H) |
| | | | | 8.21 (s, 1 H) 8.55 (t, J = 5.8 Hz, |
| | | | | 1 H) |
| 32 | 2-(7-Acetyl-1H-benzimidazol- | 391.3 | 392.0 | (DMSO-D6) δ ppm 2.56 (s, 3 H) |
| | 1-yl)-N-[3-methoxy-5- | | | 3.79 (s, 3 H) 5.34 (s, 2 H) |
| | (trifluoromethyl)phenyl]acetamide | | | 6.93 (s, 1 H) 7.32 (t, J = 7.8 Hz, |
| | | | | 1 H) 7.38-7.41 (m, J = 2.0 Hz, |
| | | | | 1 H) 7.52 (s, 1 H) 7.82 (dd, |
| | | | | J = 7.6, 1.0 Hz, 1 H) 7.92 (dd, |
| | | | | J = 8.1, 1.0 Hz, 1 H) 8.27 (s, 1 H) |
| | | | | 10.62 (s, 1 H) |
| 33 | 2-(7-Acetyl-1H-benzimidazol- | 383.4 | 384 | (400 MHz, CD₃OD) δ ppm |
| | 1-yl)-N-(3,4,5-trimethoxyphenyl) | | | 2.62 (s, 3 H), 3.72 (s, 3 H), |
| | acetamide | | | 3.78 (s, 6 H), 5.40 (s, 2 H), |
| | | | | 6.90 (s, 2 H), 7.38 (t, J = 7.8 Hz, |
| | | | | 1 H), 7.86 (dd, J = 7.6, 1.0 Hz, |
| | | | | 1 H), 7.92 (dd, J = 8.1, 1.0 Hz, |
| | | | | 1 H), 8.23 (s, 1 H) |
| 34 | 2-(7-Acetyl-1H-benzimidazol- | 329.3 | 330 | (400 MHz, CD₃OD) δ ppm |
| | 1-yl)-N-(3,4-difluoro- | | | 2.61 (s, 3 H), 5.41 (s, 2 H), |
| | phenyl)acetamide | | | 7.14-7.24 (m, 2 H), 7.40 (t, |
| | | | | J = 7.9 Hz, 1 H), 7.56-7.64 (m, |
| | | | | 1 H), 7.86 (dd, J = 7.7, 0.9 Hz, |
| | | | | 1 H), 7.92 (dd, J = 8.1, 1.0 Hz, |
| | | | | 1 H), 8.22 (s, 1 H) |
| 35 | 2-(7-Acetyl-1H-benzimidazol- | 353.4 | 354 | (400 MHz, CD₃OD) δ ppm |
| | 1-yl)-N-(3,5-dimethoxyphenyl) | | | 2.63 (s, 3 H), 3.74 (s, 6 H), |
| | acetamide | | | 5.37 (s, 2 H), 6.23 (t, J = 2.1 Hz, |
| | | | | 1 H), 6.75 (d, J = 2.3 Hz, 2 H), |
| | | | | 7.38 (t, J = 7.8 Hz, 1 H), |
| | | | | 7.85 (dd, J = 7.6, 0.8 Hz, 1 H), |
| | | | | 7.91 (dd, J = 8.0, 1.0 Hz, 1 H), |
| | | | | 8.21 (s, 1 H) |
| 36 | N-(4-tert-Butylbenzyl)-2- | 346.4 | 347 | (400 MHz, CD₃OD) δ ppm |
| | (7-cyano-1H-benzimidazol- | | | 1.29 (s, 9 H), 4.39 (s, 2 H), |
| | 1-yl)acetamide | | | 5.34 (s, 2 H), 7.24-2.29 (m, 2 H), |
| | | | | 7.33-7.38 (m, 2 H), |
| | | | | 7.42 (t, J = 8.0 Hz, 1 H), 7.71 (dd, |
| | | | | J = 7.6, 0.8 Hz, 1 H), 8.00 (dd, |
| | | | | J = 8.1, 1.0, Hz, 1 H), 8.30 (s, 1 H) |
| 37 | 2-(7-Cyano-1H-benzimidazol- | 366.4 | 367 | (400 MHz, CD₃OD) δ ppm |
| | 1-yl)-N-(3,4,5- | | | 3.72 (s, 3 H), 3.79 (s, 6 H), |
| | trimethoxyphenyl)acetamide | | | 5.47 (s, 2 H), 6.93 (s, 2 H), |
| | | | | 7.44 (t, J = 8.0 Hz, 1 H), |
| | | | | 7.71 (dd, J = 7.5, 0.8 Hz, 1 H), |
| | | | | 8.02 (dd, J = 8.2, 0.9 Hz, 1 H), |
| | | | | 8.34 (s, 1 H) |
| 38 | N-(4-Bromo-2-fluorophenyl)- | 373.2 | 373, | (400 MHz, CD₃OD) δ ppm |
| | 2-(7-cyano-1H- | | 375 | 5.53 (s, 2 H), 7.28-7.32 (m, 1 H), |
| | benzimidazol-1- | | | 7.39-7.45 (m, 2 H), |
| | yl)acetamide | | | 7.71 (dd, J = 7.6, 0.8 Hz, 1 H), |
| | | | | 7.91 (t, J = 8.6 Hz, 1 H), 8.02 (dd, |
| | | | | J = 8.4, 1.0 Hz, 1 H), 8.33 (s, 1 H) |
| 39 | 2-(7-Cyano-1H-benzimidazol- | 312.3 | 313 | (400 MHz, CD₃OD) δ ppm |
| | 1-yl)-N-(3,4-di- | | | 5.45 (s, 2 H), 7.16-7.7.28 (m, |
| | fluorophenyl)acetamide | | | 2 H), 7.43 (t, J = 8.0 Hz, 1 H), |
| | | | | 7.82-7.89 (m, 1 H), 7.71 (dd, |
| | | | | J = 7.6, 0.8 Hz, 1 H), 8.02 (dd, |
| | | | | J = 8.2, 0.9 Hz, 1 H), 8.33 (s, 1 H) |
| 40 | 2-(7-Cyano-1H-benzimidazol- | 320.4 | 321 | (400 MHz, CD₃OD) δ ppm |
| | 1-yl)-N-(3-ethoxy- | | | 1.21 (t, J = 7.0 Hz, 3 H), |
| | phenyl)acetamide | | | 3.86 (q, J = 7.0 Hz, 2 H), 5.32 (s, 2 H), |
| | | | | 6.49-6.55 (m, 1 H), |
| | | | | 6.88-6.93 (m, 1 H), 7.04 (t, J = 8.1 Hz, |
| | | | | 1 H), 7.08 (t, J = 2.1 Hz, 1 H), |
| | | | | 7.29 (t, J = 8.0 Hz, 1 H), |
| | | | | 7.57 (dd, J = 7.5, 0.8 Hz, 1 H), |
| | | | | 7.87 (dd, J = 8.1, 1.0 Hz, 1 H), |
| | | | | 8.18 (s, 1 H) |
| 41 | N-(2,3-Dihydro-1H-inden- | 368.4 | 369.2 | (CD₃CN) δ ppm |
| | 5-yl)-2-(7-pyridin-2-yl-1H- | | | 1.97-2.07 (m, 2 H) 2.77-2.85 (m, 4 H) |
| | benzimidazol-1- | | | 5.01 (s, 2 H) 6.94 (dd, J = 8.1, |
| | yl)acetamide | | | 1.5 Hz, 1 H) 7.07 (d, J = 8.1 Hz, |
| | | | | 1 H) 7.12 (s, 1 H) |
| | | | | 7.25-7.37 (m, 3 H) 7.52-7.59 (m, 1 H) |
| | | | | 7.68-7.76 (m, 1 H) |
| | | | | 7.79 (dd, J = 7.8, 1.3 Hz, 1 H) |
| | | | | 7.98 (s, 1 H) 8.62-8.68 (m, 1 H) |
| 42 | N-(4-tert-Butylbenzyl)-2- | 398.5 | 399.2 | (CD₃CN) δ ppm 1.21 (s, 9 H) |
| | (7-pyridin-2-yl-1H-benzimidazol- | | | 3.91 (d, J = 6.1 Hz, 2 H) |
| | 1-yl)acetamide | | | 4.82 (s, 2 H) 6.39 (s, 1 H) |
| | | | | 6.87-6.93 (m, 2 H) 7.16-7.27 (m, 5 H) |
| | | | | 7.40-7.45 (m, Hz, 1 H) |
| | | | | 7.64-7.71 (m, 2 H) 7.86 (s, 1 H) |
| | | | | 8.46-8.51 (m, 1 H) |
| 43 | 2-(7-Chloro-6-methoxy- | 355.8 | 356 | (400 MHz, DMSO-D6) δ ppm |
| | 1H-benzimidazol-1-yl)-N- | | | 1.95-2.02 (m, 2 H), |
| | (2,3-dihydro-1H-inden-5- | | | 2.77-2.83 (m, 4 H), 3.87 (s, 3 H), |
| | yl)acetamide | | | 5.33 (s, 2 H), 7.10 (d, J = 8.8 Hz, 1 H), |
| | | | | 7.14 (d, J = 8.1 Hz, 1 H), |
| | | | | 7.27 (d, J = 7.8 Hz, 1 H), |
| | | | | 7.47 (s, 1 H), 7.60 (d, J = 8.6 Hz, 1 H), |
| | | | | 8.13 (s, 1 H), 10.25 (s, 1 H) |
| 44 | N-[3-Methoxy-5- | 375.4 | 376 | (400 MHz, DMSO-D6) δ ppm |
| | (trifluoromethyl)phenyl]- | | | 2.37-2.44 (m, 1 H), |
| | 5,6-dihydro-4H-imidazo | | | 2.83-3.02 (m, 3 H), 3.81 (s, 3 H), 5.39 (t, |
| | [4,5,1-ij]quinoline-4- | | | J = 4.7 Hz, 1 H), 6.98 (s, 1 H), |
| | carboxamide | | | 7.02 (d, J = 7.1 Hz, 1 H), |
| | | | | 7.13 (t, J = 7.6 Hz, 1 H), |
| | | | | 7.47-7.49 (m, 2 H), 7.61 (s, 1 H), |
| | | | | 8.25 (s, 1 H), 10.76 (s, 1 H) |

### Example 45

### 2-(7-Amino-H-benzimidazol-1-yl)-N-(4-tert-butylbenzyl)acetamide

A solution of 2-(7-nitro-1H-benzimidazol-1-yl)-N-(4-tert-butylbenzyl)acetamide (0.35 g, 0.96 mmol) in methanol (15 ml) was hydrogenated in presence of Pd/C catalyst until the consumption of hydrogen ceased. The catalyst was removed by filtration through a pad of Celite™ and concentrated to yield the title compound, 0.32 mg (94%). Calculated for C₂₀H₂₄N₄O m/z: 336.44, found 337.22 [M+H]⁺. ¹H NMR (400 MHz, DMSO-D6) δ ppm 1.25 (s, 9H) 4.26 (d, J=6.1 Hz, 2H) 5.04 (s, 2H) 5.06 (s, 2H) 6.51 (dd, J=7.6, 1.0 Hz, 1H) 6.86-6.91 (m, 1H) 6.91-6.95 (m, 1H) 7.19 (d, J=8.1 Hz, 2H) 7.33 (dt, J=8.6, 2.0 Hz, 2H) 7.93 (s, 1H) 8.73 (t, J=5.6 Hz, 1H).

### Example 46

### N-(4-tert-Butylbenzyl)-2-(7-iodo-1H-benzimidazol-1-yl)acetamide

A suspension of 2-(7-amino-1H-benzimidazol-1-yl)-N-(4-tert-butylbenzyl)acetamide (30 mg, 0.09 mmol) in 2.5M H₂SO₄ (87 mL) was cooled to 0° C. and 4 M solution of NaNO₂ (25 μL) was added slowly so that the reaction temperature would not exceed 5° C. After the addition reaction mixture was kept at 0° C. for further 30 min and then added to 1.5 M solution of potassium iodide (100 μL) at ambient temperature. The resulting slurry was partitioned between ethyl acetate and aq. NaHCO₃. The organic extract was further washed with 1 M Na₂S₂O₃, water and brine, dried over Na₂SO₄ and concentrated. Purification was performed on flash silica column using ethyl acetate-methanol as the eluent.

Yield 18 mg (45%). Calculated for C₂₀H₂₂IN₃O₂ m/z: 447.31, found 448.06 [M+H]⁺. ¹H NMR (400 MHz, CD₃CN) δ ppm 1.29 (s, 9H) 4.34 (d, J=6.1 Hz, 2H) 5.21 (s, 2H) 7.00 (t, J=7.8 Hz, 1H) 7.03-7.09 (m, 1H) 7.21-7.25 (m, 2H) 7.37 (dt, J=8.6, 2.0 Hz, 2H) 7.74 (d, J=7.6 Hz, 1H) 7.77 (d, J=8.1 Hz, 1H) 8.11 (s, 1H).

### Example 47

### N-(4-tert-Butylbenzyl)-2-[7-(dimethylamino)-1H-benzimidazol-1-yl]acetamide

To a solution of 2-(7-amino-1H-benzimidazol-1-yl)-N-(4-tert-butylbenzyl)acetamide (24 mg, 66 μmol) and 37% aqueous formaldehyde (100 μL, 1.2 mmol) in ethanol (1 ml), acetic acid (60 μL) and sodium cyanoborohydride (30 mg, 0.5 mmol) were added. After 30 min the volatiles were removed under reduced pressure, and the residue was purified on preparative HPLC to yield the title compound, 15.5 mg (66%). Calculated for C₂₂H₂₈N₄O m/z: 364.23, found 365.21 [M+H]⁺. ¹H NMR (400 MHz, CD₃CN) δ ppm 1.28 (s, 9H) 2.62 (s, 6 H) 4.30 (d, J=6.1 Hz, 2H) 5.11 (s, 2H) 7.07 (m, 2H) 7.16 (m, 3H) 7.34 (m, 2H) 7.41 (dd, J=8.1, 1.0 Hz, 1H) 7.87 (s, 1H).

### Example 48

### 2-[7-(1-Hydroxy-1-methylethyl)-1H-benzimidazol-1-yl]-N-[3-methoxy-5-(trifluoromethyl)phenyl]acetamide

A solution of 2-(7-acetyl-1H-benzimidazol-1-yl)-N-[3-methoxy-5-(trifluoromethyl)phenyl]acetamide (26 mg, 0.066 mmol) in dry THF (2.5 ml) was cooled to −78° C. Methyl magnesium bromide (0.2 mL, 0.2 mmol) was added slowly over a period of 20 min and the reaction was allowed to warm up to 0° C. and kept such for additional 1 h. Reaction was quenched with aqueous semi-saturated NH₄Cl and concentrated. The residue was partitioned between ethyl acetate and 0.2 M citric acid (aq.). The organic extract was further washed with NaHCO₃, water and brine, dried over Na₂SO₄ and concentrated. Purification was performed on reversed-phase preparative HPLC.

Yield 15 mg (56%). Calculated for C₂₀H₂₀F₃N₃O₃ m/z: 407.39, found 408.03 [M+H]⁺. ¹H NMR (400 MHz, CD₃CN) δ ppm 1.57 (s, 6H) 3.71 (s, 3H) 5.51 (s, 2H) 6.84 (bs, 1H) 7.09 (t, J=7.6 Hz, 1H) 7.12-7.19 (m, 1H) 7.30-7.38 (m, 2H) 7.53 (dd, J=7.8, 1.3 Hz, 1H) 7.84 (s, 1H) 8.67 (s, 1H).

### Example 49

### N-(4-tert-Butylbenzyl)-2-[7-(1-hydroxyethyl)-1H-benzimidazol-yl]acetamide

To a solution of 2-(7-acetyl-1H-benzimidazol-1-yl)-N-(4-tert-butylbenzyl)acetamide (20 mg, 0.054 mmol) in ethanol (3 ml), sodium borohydride (10 mg) was added in single portion. After 30 min the reaction was quenched with acetic acid and concentrated to dryness. The residue was partitioned between ethyl acetate and aq. NaHCO₃. The organic extract was further washed with water and brine, dried over Na₂SO₄ and concentrated. Purification was performed on flash silica column using ethyl acetate-methanol as the eluent. Yield 20 mg (100%). Calculated for C₂₂H₂₇N₃O₂ m/z: 365.48, found 366.12 [M+H]⁺. ¹H NMR (400 MHz, DMSO-D6) δ ppm 1.26 (s, 9H) 1.40 (d, J=6.6 Hz, 3H) 4.20-4.31 (m, 2H) 5.05 (m, 1H) 5.16-5.22 (m, 1H) 5.31 (d, J=1.0 Hz, 1H) 5.32-5.37 (m, 1H) 7.15 (t, J=7.6 Hz, 1H) 7.19 (d, J=8.6 Hz, 2H) 7.27 (d, J=7.6 Hz, 1H) 7.33 (d, J=8.1 Hz, 2H) 7.54 (d, J=8.1 Hz, 1H) 8.10 (s, 1H) 8.70 (t, J=5.8 Hz, 1H).

### Example 50

### 1-{2-[(3,5-Dimethoxyphenyl)amino]-2-oxoethyl}-1H-benzimidazole-7-carboxylic acid

To [7-(methoxycarbonyl)-1H-benzimidazol-1-yl]acetic acid (0.30 g, 1.28 mmol) in DMF (6 ml) triethylamine (0.89 mL, 6.39 mmol) and 3,5-dimethoxyaniline (0.24 g, 1.54 mmol) were added followed by O-(7-azabenzotriazol-1-yl)-N,N,N′,N′-tetramethyluronium hexafluorophosphate (0.59 g, 1.54 mmol). After stirring the reaction mixture for 1 h was the volatiles were removed under reduced pressure. The residue was dissolved in a mixture of THF (10 ml) and water (3 ml) then 10% aqueous NaOH (3 ml) was added. The resulting two-phase reaction mixture was stirred intensively at ambient temperature for 5 h, diluted with water (40 ml) and 1M HCl was added to reach pH 2. Extraction with ethyl acetate:methanol 95:5 (4×50 ml), concentration of the combined organic phases and purification of the residue by column chromatography on silica using dichloromethane:methanol 9:1 as eluent afforded the title product as a white solid (0.31 g, 68%). MS (ESI) m/z: 354 [M−H]. ¹H NMR (400 MHz, CD₃OD) δ ppm 3.72 (s, 6H), 5.55 (s, 2H), 6.22 (t, J=2.2 Hz, 1H), 6.77 (d, J=2.0 Hz, 2H), 7.36 (t, J=7.8 Hz, 1H), 7.89-8.00 (m, 2H), 8.27 (s, 1H)

### Example 51

### 1-[2-(2,3-Dihydro-1H-inden-5-ylamino)-2-oxoethyl]-1H-benzimidazole-7-carboxylic acid

The title compound was synthesized from [7-(methoxycarbonyl)-1H-benzimidazol-1-yl]acetic acid and 2,3-dihydro-1H-inden-5-ylamine according to the procedure described for the preparation of 1-{2-[(3,5-dimethoxyphenyl)amino]-2-oxoethyl}-1H-benzimidazole-7-carboxylic acid affording 0.24 g (83%). MS (ESI) m/z: 336 [M+H]. ¹H NMR (400 MHz, CD₃OD) δ ppm 2.03 (m, J=7.39 Hz, 2H), 2.79-2.86 (m, 4H), 5.56 (s, 2H), 7.09 (d, J=8.1 Hz, 1H), 7.20 (dd, J=8.1, 2.0 Hz, 1H), 7.30 (t, J=7.8 Hz, 1H), 7.38 (s, 1H), 7.71-7.84 (m, 2H), 8.20 (s, 1H)

### Example 52

### N-(3,5-Dimethoxyphenyl)-2-[7-(hydroxymethyl)-1H-benzimidazol-1-yl]acetamide

To 1-{2-[(3,5-dimethoxyphenyl)amino]-2-oxoethyl}-1H-benzimidazole-7-carboxylic acid (30 mg, 0.084 mmol) in dry THF (3 ml), 2M BH₃Me₂S in THF (0.17 mL, 0.34 mmol) was added keeping the temperature at −20° C. to room temperature during a period of 27 h. The reaction mixture was quenched with acetic acid:water 1:1 (1 ml), the volatiles removed under reduced pressure and the residue purified by preparative HPLC (Xterra C8 column 19×300 mm, 0.1 M aqueous NH₄Ac/CH₃CN) giving 1.9 mg (7%) of the desired compound. MS (ESI) m/z: 342 [M+H]. ¹H NMR (400 MHz, CD₃OD) δ ppm 3.73 (s, 6H), 4.81 (s, 2H), 5.49 (s, 2H), 6.25 (t, J=2.3 Hz, 1H), 6.80 (d, J=2.3 Hz, 2H), 7.20-7.28 (m, 2H), 7.62-7.68 (m, 1H), 8.15 (s, 1H)

### Example 53

### 1-{2-[(3,5-Dimethoxyphenyl)amino]-2-oxoethyl}-N-ethyl-1H-benzimidazole-7-carboxamide

To 1-{2-[(3,5-dimethoxyphenyl)amino]-2-oxoethyl}-1H-benzimidazole-7-carboxylic acid (20 mg, 0.056 mmol) in DMF (2 ml), triethylamine (39 μL, 0.28 mmol) and i-butylchloroformate (8.8 □L, 0.068 mmol) were added. After stirring at room temperature for 10 minutes ethylammonium chloride (5.5 mg, 0.068 mmol) was added, stirring continued for 18 h and the volatiles were removed at reduced pressure. Purification by preparative HPLC (Xterra C8 column 19×300 mm, 0.1 M aqueous NH₄Ac/CH₃CN) afforded 13 mg (59%) of the title compound. MS (ESI) m/z: 383 [M+H]. ¹H NMR (400 MHz, CD₃OD), signals given for major (80%) rotamer, 6 ppm 0.86 (t, J=7.3 Hz, 3H), 2.95 (q, J=7.3 Hz, 2H), 3.77 (s, 6H), 5.17 (s, 2H), 6.30 (t, J=2.2 Hz, 1H), 6.99 (d, J=2.2 Hz, 2H), 7.36 (t, J=7.8 Hz, 1H), 7.54 (d, J=7.2 Hz, 1H), 7.84 (d, J=8.2 Hz, 1H), 8.20 (s, 1H)

### Example 54

### 1-{2-[(3,5-Dimethoxyphenyl)amino]-2-oxoethyl}-N-methyl-1H-benzimidazole-7-carboxamide

The title compound was prepared according to the procedure described for 1-{2-[(3,5-dimethoxyphenyl)amino]-2-oxoethyl}-N-ethyl-1H-benzimidazole-7-carboxamide starting from 1-{2-[(3,5-Dimethoxyphenyl)amino]-2-oxoethyl}-1H-benzimidazole-7-carboxylic acid and methylammonium chloride affording 14 mg (65%) of the targeted compound. MS (ESI) m/z: 369 [M+H]. ¹H NMR (400 MHz, CD₃OD), signals given for major (75%) rotamer, 6 ppm 2.47 (s, 3H), 3.78 (s, 6H), 5.17 (s, 2H), 6.30 (t, J=2.2 Hz, 1H), 6.98 (d, J=2.2 Hz, 2H), 7.36 (t, J=7.8 Hz, 1H), 7.54 (d, J=7.2 Hz, 1H), 7.84 (dd, J=8.1, 0.9 Hz, 1H), 8.19 (s, 1H)

### Example 55

### 1-{2-[(3,5-Dimethoxyphenyl)amino]-2-oxoethyl}-N,N-dimethyl-1H-benzimidazole-7-carboxamide

The title compound was prepared according to the procedure described for 1-{2-[(3,5-dimethoxyphenyl)amino]-2-oxoethyl}-N-ethyl-1H-benzimidazole-7-carboxamide starting from 1-{2-[(3,5-dimethoxyphenyl)amino]-2-oxoethyl}-1H-benzimidazole-7-carboxylic acid and dimethylammonium chloride affording 6.3 mg (29%) of the targeted compound. MS (ESI) m/z: 383 [M+H]. ¹H NMR (400 MHz, CD₃OD), signals given for major (70%) rotamer, 6 ppm 2.63 (s, 3H), 3.04 (s, 3H), 3.78 (s, 6H), 5.40 (s, 2H), 6.31 (t, J=2.3 Hz, 1H), 6.98 (d, J=2.3 Hz, 2H), 7.36 (t, J=7.7 Hz, 1H), 7.52 (d, J=7.3 Hz, 1H), 7.83 (dd, J=8.1, 1.0 Hz, 1H), 8.15 (s, 1H)

### Example 56

### 1-{2-[(3,5-Dimethoxyphenyl)amino]-2-oxoethyl}-N-methoxy-1H-benzimidazole-7-carboxamide

The title compound was prepared according to the procedure described for 1-{2-[(3,5-dimethoxyphenyl)amino]-2-oxoethyl}-N-ethyl-1H-benzimidazole-7-carboxamide starting from 1-{2-[(3,5-dimethoxyphenyl)amino]-2-oxoethyl}-1H-benzimidazole-7-carboxylic acid and methoxyammonium chloride affording 5.5 mg (25%) of the targeted compound. MS (ESI) m/z: 385 [M+H]. ¹H NMR (400 MHz, CD₃OD) δ ppm 3.65 (s, 3H), 3.71 (s, 6H), 5.42 (s, 2H), 6.21 (t, J=2.3 Hz, 1H), 6.77 (d, J=2.3 Hz, 2H), 7.33 (t, J=7.7 Hz, 1H), 7.39-7.44 (m, 1H), 7.86 (dd, J=8.1, 1.1 Hz, 1H), 8.23 (s, 1H)

### Example 57

### Ethyl 1-{2-[(3,5-dimethoxyphenyl)amino]-2-oxoethyl}-1H-benzimidazole-7-carboxylate

To 1-{2-[(3,5-dimethoxyphenyl)amino]-2-oxoethyl}-1H-benzimidazole-7-carboxylic acid (20 mg, 0.056 mmol) in DMF (2 ml) triethylamine (39 μL, 0.28 mmol) and O-benzotriazol-1-yl-N,N,N′,N′-tetramethyluronium hexafluorophosphate (26 mg, 0.067 mmol) were added. The resulting solution was stirred at ambient temperature for 20 minutes followed by addition of ethanol and stirring for additional 20 h. The volatiles were evaporated under reduced pressure and the residue was purified by preparative HPLC (Xterra C8 column 19×300 mm, 0.1 M aqueous NH₄Ac/CH₃CN) affording the desired product, 4.5 mg (21%). MS (ESI) m/z: 384 [M+H]. ¹H NMR (400 MHz, CD₃OD) δ ppm 1.06 (t, J=7.1 Hz, 3H), 3.78 (s, 6H), 3.98 (q, J=7.1 Hz, 2H), 5.32 (s, 2H), 6.30 (t, J=2.3 Hz, 1H), 6.97 (d, J=2.0 Hz, 2H), 7.38 (t, J=7.8 Hz, 1H), 7.58 (d, J=7.3 Hz, 1H), 7.85 (d, J=8.3 Hz, 1H), 8.20 (s, 1H)

### Example 58

### Ethyl 1-{2-[(4-tert-butylbenzyl)amino]-2-oxoethyl}-1H-benzimidazole-7-carboxylate

The title compound was prepared according to the procedure described for the preparation of ethyl 1-{2-[(3,5-dimethoxyphenyl)amino]-2-oxoethyl}-1H-benzimidazole-7-carboxylate using 1-{2-[(4-tert-butylbenzyl)amino]-2-oxoethyl}-1H-benzimidazole-7-carboxylic acid as starting material which afforded 2.2 mg (5%) of the product. MS (ESI) m/z: 394 [M+H]. ¹H NMR (400 MHz, CD₃OD) δ ppm 1.29 (s, 9H), 1.34 (t, J=7.2 Hz, 3H), 4.26 (q, J=7.2 Hz, 2H), 4.32 (s, 2H), 5.41 (s, 2H), 7.18-7.24 (m, 2H), 7.31-7.37 (m, 3H), 7.86-7.92 (m, 2H), 8.20 (s, 1H)

### Example 59

### Ethyl 1-[2-(2,3-dihydro-1H-inden-5-ylamino)-2-oxoethyl]-1H-benzimidazole-7-carboxylate

The title product was prepared according to the procedure described for the preparation of ethyl 1-{2-[(3,5-dimethoxyphenyl)amino]-2-oxoethyl}-1H-benzimidazole-7-carboxylate using 1-[2-(2,3-dihydro-1H-inden-5-ylamino)-2-oxoethyl]-1H-benzimidazole-7-carboxylic acid as starting material which afforded 6.0 mg (15%) of the product. MS (ESI) m/z: 364 [M+H]. ¹H NMR (600 MHz, CD₃OD) δ ppm 1.29 (t, J=7.1 Hz, 3H), 2.06 (m, 2H), 2.86 (m, 4H), 4.29 (q, J=7.1 Hz, 2H), 5.52 (s, 2H), 7.12 (d, J=7.9 Hz, 1H), 7.21 (d, J=8.1 Hz, 1H), 7.36 (t, J=7.8 Hz, 1H), 7.40 (s, 1H), 7.87 (d, J=7.5 Hz, 1H), 7.93 (d, J=8.0 Hz, 1H), 8.25 (s, 1H)

### Example 60

### 2-(1H-Benzimidazol-1-yl)-N-(4-tert-butylbenzyl)acetamide

The title product was prepared according to the procedure used for the preparation of compounds described in examples 1 thru 44. Calculated for C₂₀H₂₃N₃O m/z: 321.2, found 322.2 [M+H]⁺. ¹H NMR (400 MHz, DMSO-D6) δ ppm 1.26 (s, 9H) 4.26 (d, J=6.1 Hz, 2H) 4.97 (s, 2H) 7.16-7.27 (m, 4H) 7.30-7.37 (m, 2H) 7.45 (dd, J=7.1, 1.5 Hz, 1H) 7.61-7.68 (m, 1H) 8.17 (s, 1H) 8.75 (t, J=5.8 Hz, 1H)

### Example 61

### 2-(1H-Benzimidazol-1-yl)-N-(2,3-dihydro-1H-inden-5-yl)acetamide

The title product was prepared according to the procedure used for the preparation of compounds described in examples 1 thru 44. Calculated for C₂₀H₂₃N₃O m/z: 291.4, found 292 [M+H]⁺. ¹H NMR (400 MHz, DMSO-D6) δ ppm 1.92-2.03 (m, J=7.4, 2H), 2.79 (q, J=7.3 Hz, 4H), 5.13 (s, 2H), 7.14 (d, J=8.1 Hz, 1H), 7.17-7.30 (m, 3H), 7.47-7.54 (m, 2H), 7.63-7.68 (m, 1H), 8.21 (s, 1H), 10.32 (s, 1H)

### Example 62

### N-[3-Methoxy-5-(tetrahydro-2H-pyran-2-ylmethoxy)phenyl]-2-(7-nitro-1H-benzimidazol-1-yl)acetamide

Synthesised according to the general method of synthesis of the target compounds from (7-Nitro-1H-benzimidazol-1-yl)acetic acid and 3-methoxy-5-(tetrahydro-2H-pyran-2-ylmethoxy)aniline. MS (ESI) m/z 441 [M+H]. ¹H NMR (400 MHz, DMSO-D6) δ ppm 1.26-1.32 (m, 1H), 1.45-1.50 (m, 3H), 1.60-1.62 (m, 1H), 1.79-1.81 (m, 1H), 3.35-3.40 (m, partly overlapped with water peak, 1H), 3.55-3.60 (m, 1H), 3.69 (s, 3H), 3.82-3.88 (m, 3H), 5.38 (s, 2H), 6.23 (s, 1H), 6.73 (s, 1H), 6.75 (s, 1H), 7.43 (t, J=7.8 Hz, 1H), 8.03 (d, J=7.6 Hz, 1H), 8.14 (d, J=7.6 Hz, 1H), 8.45 (s, 1H), 10.34 (s, 1H).

### Example 63

### N-[3-(2-Isopropoxyethoxy)-5-methoxyphenyl]-2-(7-nitro-1H-benzimidazol-1-yl)acetamide

Synthesised according to the general method of synthesis of the target compounds from (7-Nitro-1H-benzimidazol-1-yl) acetic acid and 3-(2-isopropoxyethoxy)-5-methoxyaniline. MS (ESI) m/z 429 [M+H]. ¹H NMR (400 MHz, DMSO-D6) δ ppm 1.09 (d, J=6.1 Hz, 6H), 3.57-3.64 (m, 1H), 3.64-3.66 (m, 2H), 3.69 (s, 3H), 3.97-3.99 (m, 2H), 5.38 (s, 2H), 6.24 (t, J=2.2 Hz, 1H), 6.73 (t, J=1.8 Hz, 1H), 6.76 (t, J=1.8 Hz, 1H), 7.43 (t, J=8.1 Hz, 1H), 8.03 (d, J=7.8 Hz, 1H), 8.14 (dd, J=7.8, 0.8 Hz, 1H), 8.45 (s, 1H), 10.36 (s, 1H).

### Example 64

### N-(3-methoxy-5-[2-(2-oxopyrrolidin-1-yl)ethoxy]phenyl)-2-(7-nitro-1H-benzimidazol-1-yl)acetamide

Synthesised according to the general method of synthesis of the target compounds from (7-Nitro-1H-benzimidazol-1-yl)acetic acid and 1-[2-(3-amino-5-methoxyphenoxy)ethyl]pyrrolidin-2-one. MS (ESI) m/z 454 (M+H). ¹H NMR (400 MHz, DMSO-D6) δ ppm 1.86-1.94 (m, 2H), 2.20 (t, J=8.1 Hz, 2H), 3.42 (t, J=7.1 Hz, 2H), 3.51 (t, J=5.4 Hz, 2H), 3.70 (s, 3H), 4.00 (t, J=5.6 Hz, 2H), 5.38 (s, 2H), 6.25 (t, J=2.2 Hz, 1H), 6.73 (t, J=1.8 Hz, 1H), 6.78 (t, J=1.8 Hz, 1H), 7.43 (t, J=8.1 Hz, 1H), 8.03 (d, J=8.1 Hz, 1H), 8.14 (d, J=7.6 Hz, 1H), 8.45 (s, 1H), 10.37 (s, 1H).

### Example 65

### N-{3-[2-(]H-imidazol-1-yl)ethoxy-5-methoxyphenyl}-2-(7-nitro-1H-benzimidazol-1-yl)acetamide

Synthesised according to the general method of synthesis of the target compounds from (7-Nitro-1H-benzimidazol-1-yl)acetic acid and 3-[2-(1H-imidazol-1-yl)ethoxy]-5-methoxyaniline. MS (ESI) m/z 437 (M+H). ¹H NMR (400 MHz, DMSO-D6) δ ppm 3.69 (s, 3H), 4.16 (t, J=5.1 Hz, 2H), 4.32 (t, J=5.2 Hz, 2H), 5.38 (s, 2H), 6.23 (t, J=2.2 Hz, 1H), 6.72-6.74 (m, 1H), 6.77-6.78 (m, 1H), 6.87 (s, 1H), 7.21 (s, 1H), 7.43 (t, J=8.0 Hz, 1H), 7.65 (s, 1H), 8.03 (d, J=7.8 Hz, 1H), 8.14 (dd, J=7.8, 0.8 Hz, 1H), 8.45 (s, 1H), 10.37 (s, 1H).

Pharmacology

1. hVR1FLIPR (Fluorometric Image Plate Reader) Screening Assay

Transfected CHO cells, stably expessing hVR1 (15,000 cells/well) are seeded in 50 ul media in a black clear bottom 384 plate (Greiner) and grown in a humidified incubator (37° C., 2% CO₂), 24-30 hours prior to experiment.

Subsequently, the media is removed from the cell plate by inversion and 2 μM Fluo-4 is added using a multidrop (Labsystems). Following the 40 minutes dye incubation in the dark at 37° C. and 2% CO₂, the extracellular dye present is washed away using an EMBLA (Scatron), leaving the cells in 40 ul of assay buffer (1×HBSS, 10 mM D-Glucose, 1 mM CaCl₂, 10 mM HEPES, 10×7.5% NaHCO₃ and 2.5 mM Probenecid).

FLIPR Assay—IC₅₀ Determination Protocol

For IC₅₀ determinations the fluorescence is read using FLIPR filter 1 (em 520-545 nM). A cellular baseline recording is taken for 30 seconds, followed by a 20 μl addition of 10, titrated half-log concentrations of the test compound, yielding cellular concentration ranging from 3 μM to 0.1 nM. Data is collected every 2 seconds for a further 5 minutes prior to the addition of a VR1 agonist solution: either 50 nM solution of capsaicin or MES (2-[N-morpholino]ethanesulfonic acid) buffer (pH 5.2), by the FLIPR pipettor. The FLIPR continues to collect data for a further 4 minutes. Compounds having antagonistic properties against the hVR1 will inhibit the increase in intracellular calcium in response to the capsaicin addition. This consequently leading to a reduction in fluorescence signal and providing a reduced fluorescence reading, compared with no compound, buffer controls. Data is exported by the FLIPR program as a sum of fluorescence calculated under the curve upon the addition of capsaicin. Maximum inhibition, Hill slope and IC₅₀ data for each compound are generated.

2. DRGs were dissected out from adult Sprague Dawley rats (100-300 gr), and placed on ice in L15 Leibovitz medium. The ganglia were enzyme treated with Collagenase 80 U/ml+Dispase 34 U/ml dissolved in DMEM+5% serum, overnight at 37° C. The next day, cells were triturated with fire polished pasteur pipettes, and seeded in the center of 58 mm diameter Nunc cell dishes coated with Poly-D Lysine (1 mg/ml). The DRGs were cultured in a defined medium without foetal bovine serum, containing Dulbecco's MEM/NUT MIX F-12 (1:1) without L-glutamine but with pyridoxine, 6 mg/mL D(+)-Glucose, 100 μg/mL apo-transferrin, 1 mg/mL BSA, 20 μg/mL insulin, 2 mM L-glutamine, 50 IU/mL Penicillin, 50 μg/mL Streptomycin and 0.01 μg/mL NGF-7S.

When the cells had grown for 2 days up to 4 weeks, the experiments were done. Cells were chosen based on size and presence of neurites. Small cells with long processes were used for recording (most likely to be C neurons, with native VR1 receptors).

The cells were recorded with conventional whole cell voltage clamp patch clamp, using the following solutions (calcium ion free):

The extracellular solution comprised (in mM): NaCl 137, KCl 5, MgC₂*H₂O 1.2, HEPES 10, Glucose 10, EGTA 5, Sucrose 50, pH to 7.4 with NaOH.

The intracellular solution comprised K-gluconate 140, NaCl 3, MgCl₂*H₂O 1.2, HEPES 10, EGTA 1, pH to 7.2 with KOH. When the cells were penetrated with suction, a puff of capsaicin (500 nM) was used to determine if the cell expressed VR1 receptor. If not, a new cell was chosen. If yes, then the compounds were added in increasing doses before the capsaicin pulse (500 nM), to determine an IC₅₀ value.

### LIST OF ABBREVIATIONS

VR1 vanilloid receptor 1

IBS irritable bowel syndrome

IBD inflammatory bowel disease

GERD gastro-esophageal reflux disease

DRG Dorsal Root Ganglion

BSA Bovine Serum Albumin

HEPES 4-(2-Hydroxyethyl)piperazine-1-ethanesulfonic acid

EGTA Ethylene glycol-bis(2-aminoethylether)-N,N,N′,N′-tetraacetic acid

DMEM Dulbeccos Modified Eagle's Medium

Results

Typical IC₅₀ values as measured in the assays described above are 10 μM or less. In one aspect of the invention the IC₅₀ is below 500 nM. In another aspect of the invention the IC₅₀ is below 100 nM. In a further aspect of the invention the IC₅₀ is below 10 nM.

TABLE 1
Specimen results from the hVR1 FLIPR.

| Exam- | | |
|---|---|---|
| ple | | IC₅₀ nM |
| No. | Name | (agonist) |
| 10 | N-[3-Methoxy-5-(2-methoxyethoxy)phenyl]- | 22 (capsaicin) |
| | 2-(7-nitro-1H-benzimidazol-1-yl)acetamide | 45 (low pH) |
| 14 | 2-(7-nitro-1H-benzimidazol-1-yl)-N-(3,4,5- | 48 (capsaicin) |
| | trifluorophenyl) acetamide | 108 (low pH) |
| 32 | 2-(7-acetyl-1H-benzimidazol-1-yl)-N-[3- | 77 (capsaicin) |
| | methoxy-5-(trifluoromethyl)phenyl]acetamide | 53 (low pH) |
| 35 | 2-(7-Acetyl-1H-benzimidazol-1-yl)-N-(3,5- | 518 (capsaicin) |
| | dimethoxyphenyl) acetamide | 508 (low pH) |

1. A compound having the formula 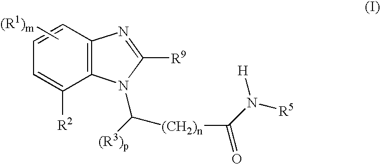
wherein:
R¹ is H, NO₂, halo, NR⁶R⁷, C₁₋₆alkyl, C₂₋₆alkenyl, C₂₋₆alkynyl, C₁₋₆haloalkyl,
C₁₋₆haloalkylO, R⁶OC₀₋₆alkyl, R⁶CO, R⁶OCO or CONR⁶R⁷;
m is 0, 1, 2 or 3;
R² is H, NO₂, halo, NR⁶R⁷, C₁₋₆alkyl, C₂₋₆alkenyl, C₂₋₆alkynyl, C₁₋₆haloalkyl, C₁₋₆haloalkylO, cyano, R⁶OC₀₋₆alkyl, R⁶CO, R⁶OCO, R⁶CONR⁷, R⁶R⁷NCO, R⁸SO₂, R⁸SO₂HN, arylC₀₋₆alkyl or heteroarylC₀₋₆alkyl;
R³ and R⁹ are each independently H or C₁₋₄alkyl;
R² and R³ optionally form a ring;
p is 0, 1 or 2;
n is 0, 2, 3 or 4;
R⁵ is C₁₋₁₀alkyl, C₆₋₁₀arylC₀₋₆alkyl, C₃₋₇cycloalkylC₀₋₆alkyl or C₅₋₆heteroarylC₀₋₆alkyl, whereby any aryl, heteroaryl or cycloalkyl may be fused with aryl, heteroaryl,
C₃₋₇cycloalkyl or C₃₋₇heterocycloalkyl, and which R⁵ may be substituted with one or more A;
A is H, OH, NO₂, cyano, R⁶CO, R⁶O(CO), halo, C₁₋₆alkyl, NR⁶R⁷, C₁₋₆haloalkyl, C₁₋₆haloalkylO, R⁶OC₀₋₆alkyl, hydroxyC₁₋₆alkyl, R⁸SO₂, R⁸SO₂HN, C₅₋₆arylO or CONR⁶R⁷;
R⁶ and R⁷ are each independently H or C₁₋₆alkyl; and
R⁸ is NR⁶R⁷ or C₁₋₄alkyl,
which compound is selected from the group consisting of
N-{3-[2-(dimethylamino)ethoxy]phenyl}-2-(7-nitro-1H-benzimidazol-1-yl)acetamide,
N-[3-(methoxymethyl)phenyl]-2-(7-nitro-1H-benzimidazol-1-yl)acetamide,
N-(1,3-dihydro-2-benzofuran-5-yl)-2-(7-nitro-1H-benzimidazol-1-yl)acetamide,
N-[3-methoxy-5-(methoxymethyl)phenyl]-2-(7-nitro-1H-benzimidazol-1-yl)acetamide,
N-[3-(methoxymethyl)-5-(trifluoromethyl)phenyl]-2-(7-nitro-1H-benzimidazol-1-yl)acetamide,
2-(7-acetyl-1H-benzimidazol-1-yl)-N-[3-(methoxymethyl)-5-(trifluoromethyl)phenyl]acetamide,
N-[3-cyano-5-(trifluoromethyl)phenyl]-2-(7-nitro-1H-benzimidazol-1-yl)acetamide,
N-[3-acetyl-5-(trifluoromethyl)phenyl]-2-(7-nitro-1H-benzimidazol-1-yl)acetamide,
2-(7-acetyl-1H-benzimidazol-1-yl)-N-[3-acetyl-5-(trifluoromethyl)phenyl]acetamide,
2-(7-acetyl-1H-benzimidazol-1-yl)-N-[3-cyano-5-(trifluoromethyl)phenyl]acetamide,
N-[3-(1-methoxyethyl)phenyl]-2-(7-nitro-1H-benzimidazol-1-yl)acetamide,
2-(7-chloro-6-methoxy-1H-benzimidazol-1-yl)-N-(2,3-dihydro-1H-inden-5-yl)acetamide,
N-[3-(2-methoxyethoxy)phenyl]-2-(7-nitro-1H-benzimidazol-1-yl)acetamide,
N-[3-methoxy-5-(2-methoxyethoxy)phenyl]-2-(7-nitro-1H-benzimidazol-1-yl)acetamide,
N-[3-(2-methoxyethoxy)-5-(trifluoromethyl)phenyl]-2-(7-nitro-1H-benzimidazol-1-yl)acetamide,
N-[3-methoxy-5-(tetrahydrofuran-2-ylmethoxy)phenyl]-2-(7-nitro-1H-benzimidazol-1-yl)acetamide,
N-[3-methoxy-5-(tetrahydrofuran-3-yloxy)phenyl]-2-(7-nitro-1H-benzimidazol-1-yl)acetamide,
N-[3-methoxy-5-(trifluoromethyl)phenyl]-5,6-dihydro-4H-imidazo[4,5,1-ij]quinoline-4-carboxamide,
2-(7-amino-1H-benzimidazol-1-yl)-N-(4-tert-butylbenzyl)acetamide,
N-(4-tert-butylbenzyl)-2-(7-iodo-1H-benzimidazol-1-yl)acetamide,
2-[7-(1-hydroxy-1-methylethyl)-1H-benzimidazol-1-yl]-N-[3-methoxy-5-(trifluoromethyl)phenyl]acetamide,
N-(4-tert-butylbenzyl)-2-[7-(1-hydroxyethyl)-1H-benzimidazol-1-yl]acetamide,
N-(2,3-dihydro-1H-inden-5-yl)-2-(7-pyridin-2-yl-1H-benzimidazol-1-yl)acetamide,
N-(4-tert-butylbenzyl)-2-(7-pyridin-2-yl-1H-benzimidazol-1-yl)acetamide,
2-(7-acetyl-1H-benzimidazol-1-yl)-N-(4-tert-butylbenzyl)acetamide,
2-(7-acetyl-1H-benzimidazol-1-yl)-N-[3-methoxy-5-(trifluoromethyl)phenyl]acetamide,
2-(7-nitro-1H-benzimidazol-1-yl)-N-(3,4,5-trifluorophenyl)acetamide,
N-(4-tert-butylbenzyl)-2-(7-cyano-1H-benzimidazol-1-yl)acetamide,
2-(7-cyano-1H-benzimidazol-1-yl)-N-(3,4,5-trimethoxyphenyl)acetamide,
N-(4-bromo-2-fluorophenyl)-2-(7-cyano-1H-benzimidazol-1-yl)acetamide,
2-(7-cyano-1H-benzimidazol-1-yl)-N-(3,4-difluorophenyl)acetamide,
2-(7-cyano-1H-benzimidazol-1-yl)-N-(3-ethoxyphenyl)acetamide,
2-(7-nitro-1H-benzimidazol-1-yl)-N-(3,4,5-trimethoxybenzyl)acetamide,
N-(3,4-difluorobenzyl)-2-(7-nitro-1H-benzimidazol-1-yl)acetamide,
N-[2-(4-methoxyphenyl)ethyl]-2-(7-nitro-1H-benzimidazol-1-yl)acetamide,
N-[2-(3-fluorophenyl)ethyl]-2-(7-nitro-1H-benzimidazol-1-yl)acetamide,
N-[2-(3-methoxyphenyl)ethyl]-2-(7-nitro-1H-benzimidazol-1-yl)acetamide,
2-(7-nitro-1H-benzimidazol-1-yl)-N-{2-[3-(trifluoromethyl)phenyl]ethyl}acetamide,
N-[2-(3,4-dimethoxyphenyl)ethyl]-2-(7-nitro-1H-benzimidazol-1-yl)acetamide,
2-(7-acetyl-1H-benzimidazol-1-yl)-N-(3,4,5-trimethoxyphenyl)acetamide,
2-(7-acetyl-1H-benzimidazol-1-yl)-N-(3,4-difluorophenyl)acetamide,
2-(7-acetyl-1H-benzimidazol-1-yl)-N-(3,5-dimethoxyphenyl)acetamide,
N-[2-(3,5-dimethoxyphenyl)ethyl]-2-(7-nitro-1H-benzimidazol-1-yl)acetamide,
N-(2,3-dihydro-1H-inden-2-yl)-2-(7-nitro-1H-benzimidazol-1-yl)acetamide,
N-[2-(5-bromo-2-methoxyphenyl)ethyl]-2-(7-nitro-1H-benzimidazol-1-yl)acetamide,
N-[1-(4-chlorobenzyl)-2-hydroxyethyl]-2-(7-nitro-1H-benzimidazol-1-yl)acetamide,
N-(2-hydroxy-2-phenylethyl)-2-(7-nitro-1H-benzimidazol-1-yl)acetamide,
1-{2-[(3,5-dimethoxyphenyl)amino]-2-oxoethyl}-1H-benzimidazole-7-carboxylic acid,
1-[2-(2,3-dihydro-1H-inden-5-ylamino)-2-oxoethyl]-1H-benzimidazole-7-carboxylic acid,
N-(3,5-dimethoxyphenyl)-2-[7-(hydroxymethyl)-1H-benzimidazol-1-yl]acetamide,
1-{2-[(3,5-dimethoxyphenyl)amino]-2-oxoethyl}-N-ethyl-1H-benzimidazole-7-carboxamide,
1-{2-[(3,5-dimethoxyphenyl)amino]-2-oxoethyl}-N-methyl-1H-benzimidazole-7-carboxamide,
1-{2-[(3,5-dimethoxyphenyl)amino]-2-oxoethyl}-N,N-dimethyl-1H-benzimidazole-7-carboxamide,
1-{2-[(3,5-dimethoxyphenyl)amino]-2-oxoethyl}-N-methoxy-1H-benzimidazole-7-carboxamide,
ethyl 1-{2-[(3,5-dimethoxyphenyl)amino]-2-oxoethyl}-1H-benzimidazole-7-carboxylate,
ethyl 1-{2-[(4-tert-butylbenzyl)amino]-2-oxoethyl}-1H-benzimidazole-7-carboxylate,
ethyl 1-[2-(2,3-dihydro-1H-inden-5-ylamino)-2-oxoethyl]-11H-benzimidazole-7-carboxylate,
N-(4-tert-butylbenzyl)-2-[7-(dimethylamino)-1H-benzimidazol-1-yl]acetamide,
N-(4-methoxy-2-naphthyl)-2-(7-nitro-1H-benzimidazol-1-yl)acetamide,
2-(1H-benzimidazol-1-yl)-N-(4-tert-butylbenzyl)acetamide,
2-(1H-benzimidazol-1-yl)-N-(2,3-dihydro-1H-inden-5-yl)acetamide,
N-(3,5-dimethoxyphenyl)-2-(7-ethynyl-1H-benzimidazol-1-yl)acetamide,
N-(3,5-dimethoxyphenyl)-2-(7-prop-1-yn-1-yl-1H-benzimidazol-1-yl)acetamide,
N-(3,5-dimethoxyphenyl)-2-[7-(1H-1,2,3-triazol-4-yl)-1H-benzimidazol-1-yl]acetamide,
N-(3,5-dimethoxyphenyl)-2-[7-(1-methyl-11H-1,2,3-triazol-4-yl)-1H-benzimidazol-1-yl]acetamide,
N-(3,5-dimethoxyphenyl)-2-[7-(1-methyl-1H-tetrazol-5-yl)-1H-benzimidazol-1-yl]acetamide,
2-(7-ethyl-1H-benzimidazol-1-yl)-N-[3-methoxy-5-(trifluoromethyl)phenyl]acetamide,
2-[7-(2-hydroxyethyl)-1H-benzimidazol-1-yl]-N-[3-methoxy-5-(trifluoromethyl)phenyl]acetamide,
2-[7-(2-hydroxy-1-methylethyl)-1H-benzimidazol-1-yl]-N-[3-methoxy-5-(trifluoromethyl)phenyl]acetamide,
N-[3-methoxy-5-(trifluoromethyl)phenyl]-2-(7-vinyl-1H-benzimidazol-1-yl)acetamide,
2-(7-isopropenyl-1H-benzimidazol-1-yl)-N-[3-methoxy-5-(trifluoro-methyl)phenyl]acetamide,
2-(7-isopropyl-1H-benzimidazol-1-yl)-N-[3-methoxy-5-(trifluoromethyl)phenyl]acetamide,
N-(3,5-dimethoxyphenyl)-2-(7-methoxy-1H-benzimidazol-1-yl)acetamide,
N-(3,5-dimethoxyphenyl)-2-(7-ethoxy-1H-benzimidazol-1-yl)acetamide,
N-(3,5-dimethoxyphenyl)-2-(7-isopropoxy-1H-benzimidazol-1-yl)acetamide,
2-(7-tert-butoxy-1H-benzimidazol-1-yl)-N-(3,5-dimethoxyphenyl)acetamide,
N-(3,5-dimethoxyphenyl)-2-[7-(trifluoromethoxy)-1H-benzimidazol-1-yl]acetamide
N-(3,5-dimethoxyphenyl)-2-[7-(methylsulfinyl)-1H-benzimidazol-1-yl]acetamide,
2-[7-(difluoromethyl)-1H-benzimidazol-1-yl]-N-(3,5-dimethoxyphenyl)acetamide,
2-[7-(cyanomethyl)-1H-benzimidazol-1-yl]-N-(3,5-dimethoxyphenyl)acetamide,
2-[7-(aminomethyl)-1H-benzimidazol-1-yl]-N-(3,5-dimethoxyphenyl)acetamide
N-(3,5-dimethoxyphenyl)-2-{7-[(dimethylamino)methyl]-1H-benzimidazol-1-yl}acetamide,
2-(7-cyclopropyl-1H-benzimidazol-1-yl)-N-(3,5-dimethoxyphenyl)acetamide,
2-(7-cyclobutyl-1H-benzimidazol-1-yl)-N-(3,5-dimethoxyphenyl)acetamide,
N-(3,5-dimethoxyphenyl)-2-[7-(methoxymethyl)-1H-benzimidazol-1-yl]acetamide,
N-(1-isopropyl-1H-benzimidazol-5-yl)-2-(7-nitro-1H-benzimidazol-1-yl)acetamide,
2-(7-fluoro-1H-benzimidazol-1-yl)-N-(1-isopropyl-1H-benzimidazol-5-yl)acetamide,
2-(7-cyano-1H-benzimidazol-1-yl)-N-(1-isopropyl-1H-benzimidazol-5-yl)acetamide,
N-(1-tert-butyl-1H-benzimidazol-5-yl)-2-(7-nitro-1H-benzimidazol-1-yl)acetamide,
N-(1-tert-butyl-2-methyl-1H-benzimidazol-5-yl)-2-(7-nitro-1H-benzimidazol-1-yl)acetamide,
N-(1-isopropyl-2-methyl-1H-benzimidazol-5-yl)-2-(7-nitro-1H-benzimidazol-1-yl)acetamide,
2-(7-cyano-1H-benzimidazol-1-yl)-N-(1-isopropyl-2-methyl-1H-benzimidazol-5-yl)acetamide,
N-(1-tert-butyl-2-methyl-1H-benzimidazol-5-yl)-2-(7-cyano-1H-benzimidazol-1-yl)acetamide,
N-(1-tert-butyl-1H-benzimidazol-5-yl)-2-(7-cyano-1H-benzimidazol-1-yl)acetamide,
N-(1-tert-butyl-1H-benzimidazol-5-yl)-2-(7-fluoro-1H-benzimidazol-1-yl)acetamide,
N-(1-tert-butyl-2-methyl-1H-benzimidazol-5-yl)-2-(7-fluoro-1H-benzimidazol-1-yl)acetamide,
2-(7-fluoro-1H-benzimidazol-1-yl)-N-(1-isopropyl-2-methyl-1H-benzimidazol-5-yl)acetamide,
N-[1-isopropyl-7-(trifluoromethyl)-1H-benzimidazol-5-yl]-2-(7-nitro-1H-benzimidazol-1-yl)acetamide,
2-(7-fluoro-1H-benzimidazol-1-yl)-N-[1-isopropyl-7-(trifluoromethyl)-1H-benzimidazol-5-yl]acetamide,
2-(7-cyano-1H-benzimidazol-1-yl)-N-[1-isopropyl-7-(trifluoromethyl)-1H-benzimidazol-5-yl]acetamide,
N-2-naphthyl-2-(7-nitro-1H-benzimidazol-1-yl)acetamide,
2-(7-cyano-1H-benzimidazol-1-yl)-N-2-naphthylacetamide,
2-(7-fluoro-1H-benzimidazol-1-yl)-N-2-naphthylacetamide,
2-(7-cyano-1H-benzimidazol-1-yl)-N-(4-methoxy-2-naphthyl)acetamide,
2-(7-fluoro-1H-benzimidazol-1-yl)-N-(4-methoxy-2-naphthyl)acetamide,
N-[3-methoxy-5-(tetrahydro-2H-pyran-2-ylmethoxy)phenyl]-2-(7-nitro-1H-benzimidazol-1-yl)acetamide,
N-[3-(2-isopropoxyethoxy)-5-methoxyphenyl]-2-(7-nitro-1H-benzimidazol-1-yl)acetamide,
N-[3,5-bis(2-ethoxyethoxy)phenyl]-2-(7-nitro-1H-benzimidazol-1-yl)acetamide,
N-{3-methoxy-5-[2-(2-oxopyrrolidin-1-yl)ethoxy]phenyl}-2-(7-nitro-1H-benzimidazol-1-yl)acetamide,
N-[3-methoxy-5-(3-morpholin-4-ylpropoxy)phenyl]-2-(7-nitro-1H-benzimidazol-1-yl)acetamide,
N,N-diethyl-2-(3-methoxy-5-{[(7-nitro-1H-benzimidazol-1-yl)acetyl]amino}phenoxy)acetamide,
N-{3-methoxy-5-[(1-methylpiperidin-2-yl)methoxy]phenyl}-2-(7-nitro-1H-benzimidazol-1-yl)acetamide,
N-{3-[2-(1H-imidazol-1-yl)ethoxy]-5-methoxyphenyl}-2-(7-nitro-1H-benzimidazol-1-yl)acetamide, and
N-{3-methoxy-5-[(1-methyl-1H-imidazol-5-yl)methoxy]phenyl}-2-(7-nitro-1H-benzimidazol-1-yl)acetamide,
or salts, solvates or solvated salts thereof.

2. A compound according to claim 1 selected from the group consisting of
N-{3-[2-(dimethylamino)ethoxy]phenyl}-2-(7-nitro-1H-benzimidazol-1-yl)acetamide,
N-[3-(methoxymethyl)phenyl]-2-(7-nitro-1H-benzimidazol-1-yl)acetamide,
N-(1,3-dihydro-2-benzofuran-5-yl)-2-(7-nitro-1H-benzimidazol-1-yl)acetamide,
N-[3-methoxy-5-(methoxymethyl)phenyl]-2-(7-nitro-1H-benzimidazol-1-yl)acetamide,
N-[3-(methoxymethyl)-5-(trifluoromethyl)phenyl]-2-(7-nitro-1H-benzimidazol-1-yl)acetamide,
2-(7-acetyl-1H-benzimidazol-1-yl)-N-[3-(methoxymethyl)-5-(trifluoromethyl)phenyl]acetamide,
N-[3-cyano-5-(trifluoromethyl)phenyl]-2-(7-nitro-1H-benzimidazol-1-yl)acetamide,
N-[3-acetyl-5-(trifluoromethyl)phenyl]-2-(7-nitro-1H-benzimidazol-1-yl)acetamide,
2-(7-acetyl-1H-benzimidazol-1-yl)-N-[3-acetyl-5-(trifluoromethyl)phenyl]acetamide,
2-(7-acetyl-1H-benzimidazol-1-yl)-N-[3-cyano-5-(trifluoromethyl)phenyl]acetamide,
N-[3-(1-methoxyethyl)phenyl]-2-(7-nitro-1H-benzimidazol-1-yl)acetamide,
2-(7-chloro-6-methoxy-1H-benzimidazol-1-yl)-N-(2,3-dihydro-1H-inden-5-yl)acetamide,
N-[3-(2-methoxyethoxy)phenyl]-2-(7-nitro-1H-benzimidazol-1-yl)acetamide,
N-[3-methoxy-5-(2-methoxyethoxy)phenyl]-2-(7-nitro-1H-benzimidazol-1-yl)acetamide,
N-[3-(2-methoxyethoxy)-5-(trifluoromethyl)phenyl]-2-(7-nitro-1H-benzimidazol-1-yl)acetamide,
N-[3-methoxy-5-(tetrahydrofuran-2-ylmethoxy)phenyl]-2-(7-nitro-1H-benzimidazol-1-yl)acetamide,
N-[3-methoxy-5-(tetrahydrofuran-3-yloxy)phenyl]-2-(7-nitro-1H-benzimidazol-1-yl)acetamide,
N-[3-methoxy-5-(trifluoromethyl)phenyl]-5,6-dihydro-4H-imidazo[4,5,1-ij]quinoline-4-carboxamide,
2-(7-amino-1H-benzimidazol-1-yl)-N-(4-tert-butylbenzyl)acetamide,
N-(4-tert-butylbenzyl)-2-(7-iodo-1H-benzimidazol-1-yl)acetamide,
2-[7-(1-hydroxy-1-methylethyl)-1H-benzimidazol-1-yl]-N-[3-methoxy-5-(trifluoromethyl)phenyl]acetamide,
N-(4-tert-butylbenzyl)-2-[7-(1-hydroxyethyl)-1H-benzimidazol-1-yl]acetamide,
N-(2,3-dihydro-1H-inden-5-yl)-2-(7-pyridin-2-yl-1H-benzimidazol-1-yl)acetamide,
N-(4-tert-butylbenzyl)-2-(7-pyridin-2-yl-1H-benzimidazol-1-yl)acetamide,
2-(7-acetyl-1H-benzimidazol-1-yl)-N-(4-tert-butylbenzyl)acetamide,
2-(7-acetyl-1H-benzimidazol-1-yl)-N-[3-methoxy-5-(trifluoromethyl)phenyl]acetamide,
2-(7-nitro-1H-benzimidazol-1-yl)-N-(3,4,5-trifluorophenyl)acetamide,
N-(4-tert-butylbenzyl)-2-(7-cyano-1H-benzimidazol-1-yl)acetamide,
2-(7-cyano-1H-benzimidazol-1-yl)-N-(3,4,5-trimethoxyphenyl)acetamide,
N-(4-bromo-2-fluorophenyl)-2-(7-cyano-1H-benzimidazol-1-yl)acetamide,
2-(7-cyano-1H-benzimidazol-1-yl)-N-(3,4-difluorophenyl)acetamide,
2-(7-cyano-1H-benzimidazol-1-yl)-N-(3-ethoxyphenyl)acetamide,
2-(7-nitro-1H-benzimidazol-1-yl)-N-(3,4,5-trimethoxybenzyl)acetamide,
N-(3,4-difluorobenzyl)-2-(7-nitro-1H-benzimidazol-1-yl)acetamide,
N-[2-(4-methoxyphenyl)ethyl]-2-(7-nitro-1H-benzimidazol-1-yl)acetamide,
N-[2-(3-fluorophenyl)ethyl]-2-(7-nitro-1H-benzimidazol-1-yl)acetamide,
N-[2-(3-methoxyphenyl)ethyl]-2-(7-nitro-1H-benzimidazol-1-yl)acetamide,
2-(7-nitro-1H-benzimidazol-1-yl)-N-{2-[3-(trifluoromethyl)phenyl]ethyl}acetamide,
N-[2-(3,4-dimethoxyphenyl)ethyl]-2-(7-nitro-1H-benzimidazol-1-yl)acetamide,
2-(7-acetyl-1H-benzimidazol-1-yl)-N-(3,4,5-trimethoxyphenyl)acetamide,
2-(7-acetyl-1H-benzimidazol-1-yl)-N-(3,4-difluorophenyl)acetamide,
2-(7-acetyl-1H-benzimidazol-1-yl)-N-(3,5-dimethoxyphenyl)acetamide,
N-[2-(3,5-dimethoxyphenyl)ethyl]-2-(7-nitro-1H-benzimidazol-1-yl)acetamide,
N-(2,3-dihydro-1H-inden-2-yl)-2-(7-nitro-1H-benzimidazol-1-yl)acetamide,
N-[2-(5-bromo-2-methoxyphenyl)ethyl]-2-(7-nitro-1H-benzimidazol-1-yl)acetamide,
N-[1-(4-chlorobenzyl)-2-hydroxyethyl]-2-(7-nitro-1H-benzimidazol-1-yl)acetamide,
N-(2-hydroxy-2-phenylethyl)-2-(7-nitro-1H-benzimidazol-1-yl)acetamide,
1-{2-[(3,5-dimethoxyphenyl)amino]-2-oxoethyl}-1H-benzimidazole-7-carboxylic acid,
1-[2-(2,3-dihydro-1H-inden-5-ylamino)-2-oxoethyl]-1H-benzimidazole-7-carboxylic acid,
N-(3,5-dimethoxyphenyl)-2-[7-(hydroxymethyl)-1H-benzimidazol-1-yl]acetamide,
1-{2-[(3,5-dimethoxyphenyl)amino]-2-oxoethyl}-N-ethyl-1H-benzimidazole-7-carboxamide,
1-{2-[(3,5-dimethoxyphenyl)amino]-2-oxoethyl}-N-methyl-1H-benzimidazole-7-carboxamide,
1-{2-[(3,5-dimethoxyphenyl)amino]-2-oxoethyl}-N,N-dimethyl-1H-benzimidazole-7-carboxamide,
1-{2-[(3,5-dimethoxyphenyl)amino]-2-oxoethyl}-N-methoxy-1H-benzimidazole-7-carboxamide,
ethyl 1-{2-[(3,5-dimethoxyphenyl)amino]-2-oxoethyl}-1H-benzimidazole-7-carboxylate,
ethyl 1-{2-[(4-tert-butylbenzyl)amino]-2-oxoethyl}-1H-benzimidazole-7-carboxylate,
ethyl 1-[2-(2,3-dihydro-1H-inden-5-ylamino)-2-oxoethyl]-11H-benzimidazole-7-carboxylate,
N-(4-tert-butylbenzyl)-2-[7-(dimethylamino)-1H-benzimidazol-1-yl]acetamide,
N-(4-methoxy-2-naphthyl)-2-(7-nitro-1H-benzimidazol-1-yl)acetamide,
2-(1H-benzimidazol-1-yl)-N-(4-tert-butylbenzyl)acetamide, and
2-(1H-benzimidazol-1-yl)-N-(2,3-dihydro-1H-inden-5-yl)acetamide,
N-(3,5-dimethoxyphenyl)-2-(7-ethynyl-1H-benzimidazol-1-yl)acetamide,
N-(3,5-dimethoxyphenyl)-2-(7-prop-1-yn-1-yl-1H-benzimidazol-1-yl)acetamide,
N-(3,5-dimethoxyphenyl)-2-[7-(1H-1,2,3-triazol-4-yl)-1H-benzimidazol-1-yl]acetamide,
N-(3,5-dimethoxyphenyl)-2-[7-(1-methyl-11H-1,2,3-triazol-4-yl)-1H-benzimidazol-1-yl]acetamide,
N-(3,5-dimethoxyphenyl)-2-[7-(1-methyl-1H-tetrazol-5-yl)-1H-benzimidazol-1-yl]acetamide,
2-(7-ethyl-1H-benzimidazol-1-yl)-N-[3-methoxy-5-(trifluoromethyl)phenyl]acetamide,
2-[7-(2-hydroxyethyl)-1H-benzimidazol-1-yl]-N-[3-methoxy-5-(trifluoromethyl)phenyl]acetamide,
2-[7-(2-hydroxy-1-methylethyl)-1H-benzimidazol-1-yl]-N-[3-methoxy-5-(trifluoromethyl)phenyl]acetamide,
N-[3-methoxy-5-(trifluoromethyl)phenyl]-2-(7-vinyl-1H-benzimidazol-1-yl)acetamide,
2-(7-isopropenyl-1H-benzimidazol-1-yl)-N-[3-methoxy-5-(trifluoromethyl)phenyl]acetamide,
2-(7-isopropyl-1H-benzimidazol-1-yl)-N-[3-methoxy-5-(trifluoromethyl)phenyl]acetamide,
N-(3,5-dimethoxyphenyl)-2-(7-methoxy-1H-benzimidazol-1-yl)acetamide,
N-(3,5-dimethoxyphenyl)-2-(7-ethoxy-1H-benzimidazol-1-yl)acetamide,
N-(3,5-dimethoxyphenyl)-2-(7-isopropoxy-1H-benzimidazol-1-yl)acetamide,
2-(7-tert-butoxy-1H-benzimidazol-1-yl)-N-(3,5-dimethoxyphenyl)acetamide,
N-(3,5-dimethoxyphenyl)-2-[7-(trifluoromethoxy)-1H-benzimidazol-1-yl]acetamide
N-(3,5-dimethoxyphenyl)-2-[7-(methylsulfinyl)-1H-benzimidazol-1-yl]acetamide,
2-[7-(difluoromethyl)-1H-benzimidazol-1-yl]-N-(3,5-dimethoxyphenyl)acetamide,
2-[7-(cyanomethyl)-1H-benzimidazol-1-yl]-N-(3,5-dimethoxyphenyl)acetamide,
2-[7-(aminomethyl)-1H-benzimidazol-1-yl]-N-(3,5-dimethoxyphenyl)acetamide
N-(3,5-dimethoxyphenyl)-2-{7-[(dimethylamino)methyl]-1H-benzimidazol-1-yl}acetamide,
2-(7-cyclopropyl-1H-benzimidazol-1-yl)-N-(3,5-dimethoxyphenyl)acetamide,
2-(7-cyclobutyl-11H-benzimidazol-1-yl)-N-(3,5-dimethoxyphenyl)acetamide,
N-(3,5-dimethoxyphenyl)-2-[7-(methoxymethyl)-1H-benzimidazol-1-yl]acetamide,
N-(1-isopropyl-1H-benzimidazol-5-yl)-2-(7-nitro-1H-benzimidazol-1-yl)acetamide,
2-(7-fluoro-1H-benzimidazol-1-yl)-N-(1-isopropyl-1H-benzimidazol-5-yl)acetamide,
2-(7-cyano-1H-benzimidazol-1-yl)-N-(1-isopropyl-1H-benzimidazol-5-yl)acetamide,
N-(1-tert-butyl-1H-benzimidazol-5-yl)-2-(7-nitro-1H-benzimidazol-1-yl)acetamide,
N-(1-tert-butyl-2-methyl-1H-benzimidazol-5-yl)-2-(7-nitro-1H-benzimidazol-1-yl)acetamide,
N-(1-isopropyl-2-methyl-1H-benzimidazol-5-yl)-2-(7-nitro-1H-benzimidazol-1-yl)acetamide,
2-(7-cyano-1H-benzimidazol-1-yl)-N-(1-isopropyl-2-methyl-1H-benzimidazol-5-yl)acetamide,
N-(1-tert-butyl-2-methyl-1H-benzimidazol-5-yl)-2-(7-cyano-1H-benzimidazol-1-yl)acetamide,
N-(1-tert-butyl-1H-benzimidazol-5-yl)-2-(7-cyano-1H-benzimidazol-1-yl)acetamide,
N-(1-tert-butyl-1H-benzimidazol-5-yl)-2-(7-fluoro-1H-benzimidazol-1-yl)acetamide,
N-(1-tert-butyl-2-methyl-1H-benzimidazol-5-yl)-2-(7-fluoro-1H-benzimidazol-1-yl)acetamide,
2-(7-fluoro-1H-benzimidazol-1-yl)-N-(1-isopropyl-2-methyl-1H-benzimidazol-5-yl)acetamide,
N-[1-isopropyl-7-(trifluoromethyl)-1H-benzimidazol-5-yl]-2-(7-nitro-1H-benzimidazol-1-yl)acetamide,
2-(7-fluoro-1H-benzimidazol-1-yl)-N-[1-isopropyl-7-(trifluoromethyl)-1H-benzimidazol-5-yl]acetamide,
2-(7-cyano-1H-benzimidazol-1-yl)-N-[1-isopropyl-7-(trifluoromethyl)-1H-benzimidazol-5-yl]acetamide,
N-2-naphthyl-2-(7-nitro-1H-benzimidazol-1-yl)acetamide,
2-(7-cyano-1H-benzimidazol-1-yl)-N-2-naphthylacetamide,
2-(7-fluoro-1H-benzimidazol-1-yl)-N-2-naphthylacetamide,
2-(7-cyano-1H-benzimidazol-1-yl)-N-(4-methoxy-2-naphthyl)acetamide,
2-(7-fluoro-1H-benzimidazol-1-yl)-N-(4-methoxy-2-naphthyl)acetamide,
N-[3-methoxy-5-(tetrahydro-2H-pyran-2-ylmethoxy)phenyl]-2-(7-nitro-1H-benzimidazol-1-yl)acetamide,
N-[3-(2-isopropoxyethoxy)-5-methoxyphenyl]-2-(7-nitro-1H-benzimidazol-1-yl)acetamide, and
N-[3-methoxy-5-(3-morpholin-4-ylpropoxy)phenyl]-2-(7-nitro-1H-benzimidazol-1-yl)acetamide,
or salts, solvates or solvated salts thereof.

3. A compound according to claim 1 selected from the group consisting of
N-{3-[2-(dimethylamino)ethoxy]phenyl}-2-(7-nitro-1H-benzimidazol-1-yl)acetamide,
N-[3-(methoxymethyl)phenyl]-2-(7-nitro-1H-benzimidazol-1-yl)acetamide,
N-(1,3-dihydro-2-benzofuran-5-yl)-2-(7-nitro-1H-benzimidazol-1-yl)acetamide,
N-[3-methoxy-5-(methoxymethyl)phenyl]-2-(7-nitro-1H-benzimidazol-1-yl)acetamide,
N-[3-(methoxymethyl)-5-(trifluoromethyl)phenyl]-2-(7-nitro-1H-benzimidazol-1-yl)acetamide,
2-(7-acetyl-1H-benzimidazol-1-yl)-N-[3-(methoxymethyl)-5-(trifluoromethyl)phenyl]acetamide,
N-[3-cyano-5-(trifluoromethyl)phenyl]-2-(7-nitro-1H-benzimidazol-1-yl)acetamide,
N-[3-acetyl-5-(trifluoromethyl)phenyl]-2-(7-nitro-1H-benzimidazol-1-yl)acetamide,
2-(7-acetyl-1H-benzimidazol-1-yl)-N-[3-acetyl-5-(trifluoromethyl)phenyl]acetamide,
2-(7-acetyl-1H-benzimidazol-1-yl)-N-[3-cyano-5-(trifluoromethyl)phenyl]acetamide,
N-[3-(1-methoxyethyl)phenyl]-2-(7-nitro-1H-benzimidazol-1-yl)acetamide,
2-(7-chloro-6-methoxy-1H-benzimidazol-1-yl)-N-(2,3-dihydro-1H-inden-5-yl)acetamide,
N-[3-(2-methoxyethoxy)phenyl]-2-(7-nitro-1H-benzimidazol-1-yl)acetamide,
N-[3-methoxy-5-(2-methoxyethoxy)phenyl]-2-(7-nitro-1H-benzimidazol-1-yl)acetamide,
N-[3-(2-methoxyethoxy)-5-(trifluoromethyl)phenyl]-2-(7-nitro-1H-benzimidazol-1-yl)acetamide,
N-[3-methoxy-5-(tetrahydrofuran-2-ylmethoxy)phenyl]-2-(7-nitro-1H-benzimidazol-1-yl)acetamide,
N-[3-methoxy-5-(tetrahydrofuran-3-yloxy)phenyl]-2-(7-nitro-1H-benzimidazol-1-yl)acetamide,
N-[3-methoxy-5-(trifluoromethyl)phenyl]-5,6-dihydro-4H-imidazo[4,5,1-ij]quinoline-4-carboxamide,
2-(7-amino-1H-benzimidazol-1-yl)-N-(4-tert-butylbenzyl)acetamide,
N-(4-tert-butylbenzyl)-2-(7-iodo-1H-benzimidazol-1-yl)acetamide,
2-[7-(1-hydroxy-1-methylethyl)-1H-benzimidazol-1-yl]-N-[3-methoxy-5-(trifluoromethyl)phenyl]acetamide,
N-(4-tert-butylbenzyl)-2-[7-(1-hydroxyethyl)-1H-benzimidazol-1-yl]acetamide,
N-(2,3-dihydro-1H-inden-5-yl)-2-(7-pyridin-2-yl-1H-benzimidazol-1-yl)acetamide,
N-(4-tert-butylbenzyl)-2-(7-pyridin-2-yl-1H-benzimidazol-1-yl)acetamide,
2-(7-acetyl-1H-benzimidazol-1-yl)-N-(4-tert-butylbenzyl)acetamide,
2-(7-acetyl-1H-benzimidazol-1-yl)-N-[3-methoxy-5-(trifluoromethyl)phenyl]acetamide,
2-(7-nitro-1H-benzimidazol-1-yl)-N-(3,4,5-trifluorophenyl)acetamide,
N-(4-tert-butylbenzyl)-2-(7-cyano-1H-benzimidazol-1-yl)acetamide,
2-(7-cyano-1H-benzimidazol-1-yl)-N-(3,4,5-trimethoxyphenyl)acetamide,
N-(4-bromo-2-fluorophenyl)-2-(7-cyano-1H-benzimidazol-1-yl)acetamide,
2-(7-cyano-1H-benzimidazol-1-yl)-N-(3,4-difluorophenyl)acetamide,
2-(7-cyano-1H-benzimidazol-1-yl)-N-(3-ethoxyphenyl)acetamide,
2-(7-nitro-1H-benzimidazol-1-yl)-N-(3,4,5-trimethoxybenzyl)acetamide,
N-(3,4-difluorobenzyl)-2-(7-nitro-1H-benzimidazol-1-yl)acetamide,
N-[2-(4-methoxyphenyl)ethyl]-2-(7-nitro-1H-benzimidazol-1-yl)acetamide,
N-[2-(3-fluorophenyl)ethyl]-2-(7-nitro-1H-benzimidazol-1-yl)acetamide,
N-[2-(3-methoxyphenyl)ethyl]-2-(7-nitro-1H-benzimidazol-1-yl)acetamide,
2-(7-nitro-1H-benzimidazol-1-yl)-N-{2-[3-(trifluoromethyl)phenyl]ethyl}acetamide,
N-[2-(3,4-dimethoxyphenyl)ethyl]-2-(7-nitro-1H-benzimidazol-1-yl)acetamide,
2-(7-acetyl-1H-benzimidazol-1-yl)-N-(3,4,5-trimethoxyphenyl)acetamide,
2-(7-acetyl-1H-benzimidazol-1-yl)-N-(3,4-difluorophenyl)acetamide,
2-(7-acetyl-1H-benzimidazol-1-yl)-N-(3,5-dimethoxyphenyl)acetamide,
N-[2-(3,5-dimethoxyphenyl)ethyl]-2-(7-nitro-1H-benzimidazol-1-yl)acetamide,
N-(2,3-dihydro-1H-inden-2-yl)-2-(7-nitro-1H-benzimidazol-1-yl)acetamide,
N-[2-(5-bromo-2-methoxyphenyl)ethyl]-2-(7-nitro-1H-benzimidazol-1-yl)acetamide,
N-[1-(4-chlorobenzyl)-2-hydroxyethyl]-2-(7-nitro-1H-benzimidazol-1-yl)acetamide,
N-(2-hydroxy-2-phenylethyl)-2-(7-nitro-1H-benzimidazol-1-yl)acetamide,
1-{2-[(3,5-dimethoxyphenyl)amino]-2-oxoethyl}-1H-benzimidazole-7-carboxylic acid,
1-[2-(2,3-dihydro-1H-inden-5-ylamino)-2-oxoethyl]-1H-benzimidazole-7-carboxylic acid,
N-(3,5-dimethoxyphenyl)-2-[7-(hydroxymethyl)-1H-benzimidazol-1-yl]acetamide,
1-{2-[(3,5-dimethoxyphenyl)amino]-2-oxoethyl}-N-ethyl-1H-benzimidazole-7-carboxamide,
1-{2-[(3,5-dimethoxyphenyl)amino]-2-oxoethyl}-N-methyl-1H-benzimidazole-7-carboxamide,
1-{2-[(3,5-dimethoxyphenyl)amino]-2-oxoethyl}-N,N-dimethyl-1H-benzimidazole-7-carboxamide,
1-{2-[(3,5-dimethoxyphenyl)amino]-2-oxoethyl}-N-methoxy-1H-benzimidazole-7-carboxamide,
ethyl 1-{2-[(3,5-dimethoxyphenyl)amino]-2-oxoethyl}-1H-benzimidazole-7-carboxylate,
ethyl 1-{2-[(4-tert-butylbenzyl)amino]-2-oxoethyl}-1H-benzimidazole-7-carboxylate,
ethyl 1-[2-(2,3-dihydro-1H-inden-5-ylamino)-2-oxoethyl]-11H-benzimidazole-7-carboxylate,
N-(4-tert-butylbenzyl)-2-[7-(dimethylamino)-1H-benzimidazol-1-yl]acetamide,
N-(4-methoxy-2-naphthyl)-2-(7-nitro-1H-benzimidazol-1-yl)acetamide,
2-(1H-benzimidazol-1-yl)-N-(4-tert-butylbenzyl)acetamide, and
2-(1H-benzimidazol-1-yl)-N-(2,3-dihydro-1H-inden-5-yl)acetamide, or salts, solvates or solvated salts thereof.

4. A compound according to claim 1 selected from the group consisting of
N-(3,5-dimethoxyphenyl)-2-(7-ethynyl-1H-benzimidazol-1-yl)acetamide,
N-(3,5-dimethoxyphenyl)-2-(7-prop-1-yn-1-yl-1H-benzimidazol-1-yl)acetamide,
N-(3,5-dimethoxyphenyl)-2-[7-(1H-1,2,3-triazol-4-yl)-1H-benzimidazol-1-yl]acetamide,
N-(3,5-dimethoxyphenyl)-2-[7-(1-methyl-11H-1,2,3-triazol-4-yl)-1H-benzimidazol-1-yl]acetamide,
N-(3,5-dimethoxyphenyl)-2-[7-(1-methyl-1H-tetrazol-5-yl)-1H-benzimidazol-1-yl]acetamide,
2-(7-ethyl-1H-benzimidazol-1-yl)-N-[3-methoxy-5-(trifluoromethyl)phenyl]acetamide,
2-[7-(2-hydroxyethyl)-1H-benzimidazol-1-yl]-N-[3-methoxy-5-(trifluoromethyl)phenyl]acetamide,
2-[7-(2-hydroxy-1-methylethyl)-1H-benzimidazol-1-yl]-N-[3-methoxy-5-(trifluoromethyl)phenyl]acetamide,
N-[3-methoxy-5-(trifluoromethyl)phenyl]-2-(7-vinyl-1H-benzimidazol-1-yl)acetamide,
2-(7-isopropenyl-1H-benzimidazol-1-yl)-N-[3-methoxy-5-(trifluoromethyl)phenyl]acetamide,
2-(7-isopropyl-1H-benzimidazol-1-yl)-N-[3-methoxy-5-(trifluoromethyl)phenyl]acetamide,
N-(3,5-dimethoxyphenyl)-2-(7-methoxy-1H-benzimidazol-1-yl)acetamide,
N-(3,5-dimethoxyphenyl)-2-(7-ethoxy-1H-benzimidazol-1-yl)acetamide,
N-(3,5-dimethoxyphenyl)-2-(7-isopropoxy-1H-benzimidazol-1-yl)acetamide,
2-(7-tert-butoxy-1H-benzimidazol-1-yl)-N-(3,5-dimethoxyphenyl)acetamide,
N-(3,5-dimethoxyphenyl)-2-[7-(trifluoromethoxy)-1H-benzimidazol-1-yl]acetamide
N-(3,5-dimethoxyphenyl)-2-[7-(methylsulfinyl)-1H-benzimidazol-1-yl]acetamide,
2-[7-(difluoromethyl)-1H-benzimidazol-1-yl]-N-(3,5-dimethoxyphenyl)acetamide,
2-[7-(cyanomethyl)-1H-benzimidazol-1-yl]-N-(3,5-dimethoxyphenyl)acetamide,
2-[7-(aminomethyl)-1H-benzimidazol-1-yl]-N-(3,5-dimethoxyphenyl)acetamide
N-(3,5-dimethoxyphenyl)-2-{7-[(dimethylamino)methyl]-1H-benzimidazol-1-yl}acetamide,
2-(7-cyclopropyl-1H-benzimidazol-1-yl)-N-(3,5-dimethoxyphenyl)acetamide,
2-(7-cyclobutyl-11H-benzimidazol-1-yl)-N-(3,5-dimethoxyphenyl)acetamide,
N-(3,5-dimethoxyphenyl)-2-[7-(methoxymethyl)-1H-benzimidazol-1-yl]acetamide,
N-(1-isopropyl-1H-benzimidazol-5-yl)-2-(7-nitro-1H-benzimidazol-1-yl)acetamide,
2-(7-fluoro-1H-benzimidazol-1-yl)-N-(1-isopropyl-1H-benzimidazol-5-yl)acetamide,
2-(7-cyano-1H-benzimidazol-1-yl)-N-(1-isopropyl-1H-benzimidazol-5-yl)acetamide,
N-(1-tert-butyl-1H-benzimidazol-5-yl)-2-(7-nitro-1H-benzimidazol-1-yl)acetamide,
N-(1-tert-butyl-2-methyl-1H-benzimidazol-5-yl)-2-(7-nitro-1H-benzimidazol-1-yl)acetamide,
N-(1-isopropyl-2-methyl-1H-benzimidazol-5-yl)-2-(7-nitro-1H-benzimidazol-1-yl)acetamide,
2-(7-cyano-1H-benzimidazol-1-yl)-N-(1-isopropyl-2-methyl-1H-benzimidazol-5-yl)acetamide,
N-(1-tert-butyl-2-methyl-1H-benzimidazol-5-yl)-2-(7-cyano-1H-benzimidazol-1-yl)acetamide,
N-(1-tert-butyl-1H-benzimidazol-5-yl)-2-(7-cyano-1H-benzimidazol-1-yl)acetamide,
N-(1-tert-butyl-1H-benzimidazol-5-yl)-2-(7-fluoro-1H-benzimidazol-1-yl)acetamide,
N-(1-tert-butyl-2-methyl-1H-benzimidazol-5-yl)-2-(7-fluoro-1H-benzimidazol-1-yl)acetamide,
2-(7-fluoro-1H-benzimidazol-1-yl)-N-(1-isopropyl-2-methyl-1H-benzimidazol-5-yl)acetamide,
N-[1-isopropyl-7-(trifluoromethyl)-1H-benzimidazol-5-yl]-2-(7-nitro-1H-benzimidazol-1-yl)acetamide,
2-(7-fluoro-1H-benzimidazol-1-yl)-N-[1-isopropyl-7-(trifluoromethyl)-1H-benzimidazol-5-yl]acetamide,
2-(7-cyano-1H-benzimidazol-1-yl)-N-[1-isopropyl-7-(trifluoromethyl)-1H-benzimidazol-5-yl]acetamide,
N-2-naphthyl-2-(7-nitro-1H-benzimidazol-1-yl)acetamide,
2-(7-cyano-1H-benzimidazol-1-yl)-N-2-naphthylacetamide,
2-(7-fluoro-1H-benzimidazol-1-yl)-N-2-naphthylacetamide,
2-(7-cyano-1H-benzimidazol-1-yl)-N-(4-methoxy-2-naphthyl)acetamide,
2-(7-fluoro-1H-benzimidazol-1-yl)-N-(4-methoxy-2-naphthyl)acetamide,
N-[3-methoxy-5-(tetrahydro-2H-pyran-2-ylmethoxy)phenyl]-2-(7-nitro-1H-benzimidazol-1-yl)acetamide,
N-[3-(2-isopropoxyethoxy)-5-methoxyphenyl]-2-(7-nitro-1H-benzimidazol-1-yl)acetamide, N-[3,5-bis(2-ethoxyethoxy)phenyl]-2-(7-nitro-1H-benzimidizol-1-yl)acetamide, N-{3-methoxy-5-[2-(2-oxopyrrolidin-1-yl)ethoxy]phenyl}-2-(7-nitro-1H-benzimidazol-1-yl)acetamide,
N-[3-methoxy-5-(3-morpholin-4-ylpropoxy)phenyl]-2-(7-nitro-1H-benzimidazol-1-yl)acetamide, N,N-diethyl-2-(3-methoxy-5-{[(7-nitro-1H-benzimidazol-1-yl)acetyl]amino}phenoxy)acetamide,
N-{3-methoxy-5-[(1-methylpiperidin-2-yl)methoxy]phenyl}-2-(7-nitro-1H-benzimidazol-1-yl)acetamide,
N-{3-[2-(1H-imidazol-1-yl)ethoxy]-5-methoxyphenyl}-2-(7-nitro-1H-benzimidazol-1-yl)acetamide, and
N-{3-methoxy-5-[(1-methyl-1H-imidazol-5-yl)methoxy]phenyl}-2-(7-nitro-1H-benzimidazol-1-yl)acetamide,
or salts, solvates or solvated salts thereof.

5. A pharmaceutical composition comprising as active ingredient a therapeutically effective amount of the compound according to claim 1, in association with one or more pharmaceutically acceptable diluents, excipients and/or inert carriers.

6. A method of treating a VR1 mediated disorder, the method comprising administering to a subject the composition according to claim 5.

7. (canceled)

8. A method of treatment of a VR1 mediated disorder, the method comprising administering to a subject a compound according to claim 1.

9. The method according to claim 8, wherein the VR1 mediated disorder is an acute or chronic pain disorder.

10. The method according to claim 8 wherein the VR1 mediated disorder comprises acute or chronic neuropathic pain.

11. The method according to claim 8 wherein the VR1 mediated disorder comprises acute or chronic inflammatory pain.

12. The method according to claim 8 wherein the VR1 mediated disorder comprises low back pain, post-operative pain, visceral pains like chronic pelvic pain, cystitis, including interstitial cystitis and pain related thereto, ischeamic, sciatia, diabetic neuropathy, multiple sclerosis, arthritis, fibromyalgia, psoriasis, cancer, emesis, urinary incontinence, hyperactive bladder, HIV neuropathy, gastro-esophageal reflux disease (GERD), irritable bowel syndrome (IBS), inflammatory bowel disease (IBD) or pancreatitis.

13. The method according to claim 8 wherein the VR1 mediated disorder comprises a respiratory disease.

14. A method of treatment of VR1 mediated disorders and for treatment of acute and chronic pain disorders, acute and chronic neuropathic pain and acute and chronic inflammatory pain, and respiratory diseases, comprising administering to a mammal, including man in need of such treatment, a therapeutically effective amount of a compound according to claim 1.

15. A compound selected from the group consisting of
3-methoxy-5-(methoxymethyl)aniline,
3-(methoxymethyl)-5-(trifluoromethyl)aniline,
1-(methoxymethyl)-3-nitro-5-(trifluoromethyl)benzene,
1-[3-amino-5-(trifluoromethyl)phenyl]ethanone,
(7-chloro-6-methoxy-1H-benzimidazol-1-yl)acetic acid,
2-[(2-chloro-3-methoxy-6-nitrophenyl)amino]ethanol,
2-(7-chloro-6-methoxy-1H-benzimidazol-1-yl)ethanol,
3-(2-methoxyethoxy)-5-(trifluoromethyl)aniline,
1-(2-methoxyethoxy)-3-nitro-5-(trifluoromethyl)benzene,
3-methoxy-5-(tetrahydrofuran-2-ylmethoxy)aniline,
2-[(3-methoxy-5-nitrophenoxy)methyl]tetrahydrofuran,
3-methoxy-5-(tetrahydrofuran-3-yloxy)aniline,
3-(3-methoxy-5-nitrophenoxy)tetrahydrofuran,
5,6-dihydro-4H-imidazo[4,5,1-ij]quinoline-4-carboxylic acid,
methyl 8-amino-1,2,3,4-tetrahydroquinoline-2-carboxylate,
(7-pyridin-2-yl-1H-benzimidazol-1-yl)acetic acid,
methyl (7-bromo-1H-benzimidazol-1-yl)acetate,
methyl (7-pyridin-2-yl-1H-benzimidazol-1-yl)acetate,
3-methoxy-5-(tetrahydro-2H-pyran-2-ylmethoxy)aniline, and
3-(2-isopropoxyethoxy)-5-methoxyaniline,

16. (canceled)

17. The method of claim 6, wherein the VR1 mediated disorder is an acute or chronic pain disorder.

18. The method of claim 6, wherein the VR1 mediated disorder comprises acute or chronic neuropathic pain.

19. The method of claim 6, wherein the VR1 mediated disorder comprises acute or chronic inflammatory pain.

20. The method of claim 6, wherein the VR1 mediated disorder comprises a respiratory disease.
